(19) United States
(12) Patent Application Publication
(10) Pub. No.: US 2023/0242944 A1
(43) Pub. Date: Aug. 3, 2023

(54) METHODS FOR PRODUCTION OF DIATRAEA SACCHARALIS PHEROMONE PRECURSORS

(71) Applicants: Jan Christer Lofstedt, Lund (SE); Per Fredrik Hofvander, Bjärred (SE); Honglei Wang, Lund (SE); Baojian Ding, Lund (SE); Marie Dam, Birkeröd (DK); ISCA Technologies, Inc., Riverside, CA (US)

(72) Inventors: Jan Christer Lofstedt, Lund (SE); Per Fredrik Hofvander, Bjärred (SE); Honglei Wang, Lund (SE); Baojian Ding, Lund (SE); Marie Dam, Birkeröd (DK); Agenor Mafra-Neto, Riverside, CA (US)

(21) Appl. No.: 18/161,745

(22) Filed: Jan. 30, 2023

Related U.S. Application Data
(60) Provisional application No. 63/305,088, filed on Jan. 31, 2022.

(51) Int. Cl. C12P 7/04; C12N 9/02; C12N 15/52

(52) U.S. Cl. CPC C12P 7/04; C12N 9/0008; C12N 9/0071; C12N 15/52

(57) ABSTRACT

The present invention relates to methods of producing Diatraea saccharalis pheromone precursors and genetically modified plants and microorganisms capable of producing Diatraea saccharalis pheromone precursors. The genetically modified plants and microorganisms include a heterologous gene encoding a first fatty-acyl desaturase, a second fatty-acyl desaturase, and a fatty-acyl reductase.

### CROSS-REFERENCE TO RELATED APPLICATIONS

This application claims the benefit of U.S. Provisional Application No. 63/305,088, filed on Jan. 31, 2022, the teachings of which are expressly incorporated by reference.

### STATEMENT RE: FEDERALLY SPONSORED RESEARCH/DEVELOPMENT

Not Applicable

### REFERENCE TO AN ELECTRONIC SEQUENCE LISTING

The contents of the electronic sequence listing (ISCA_041US.xml; Size 54,247 bytes; and Date of Creation: Jan. 30, 2022) is herein incorporated by reference in its entirety.

### BACKGROUND

### Technical Field

The present disclosure relates generally to a process for preparing insect pheromones in plants and microorganisms, and more particularly, insect pheromones of Diatraea saccharalis in plants and microorganisms.

### Description of the Prior Art

The sugarcane borer Diatraea saccharalis (Fabricius) (Lepidoptera: Crambidae) is widely distributed throughout southern USA, Central America, and the tropical and subtropical zones of South America, it's dispersal likely mediated by human migration and trade. It's a key pest on sugarcane and maize and uses other grasses as hosts. The larvae cause damage by eating leaves and boring into stalks, which in sugarcane production where 19-25% of internodes are bored can diminish sugar yields by 8-20%. The estimated annual economic loss due to pest insects on sugarcane (in addition to D. saccharalis, other moth genera such as Chilo, Sesamia, and Scirpophaga also cause damage to sugarcane) is more than US$4.5 billion in Brazil alone, the world's largest sugarcane producer.

Insecticidal control of D. saccharalis is not efficient because the larval and pupal stages are protected inside the plant, and also because all developmental stages are present throughout the year, but integrated pest management (IPM), where the focus lies on monitoring, prevention and limited use of pesticides, optimised cultural practices using resistant cultivars, and biological control or the use of sex pheromones may provide a solution.

Pheromones are environmentally friendly alternatives to the use of traditional pesticides for the control of insect pests. For this purpose, synthetic pheromones are produced annually in large quantities. The use of pheromones for the control of pest insects has many advantages over the use of conventional chemical-based pesticides.

Pheromones are non-toxic. They have no adverse effects on non-target organisms, and do not kill parasitoids or other beneficial insects. The risks of resistance being developed in the pests are small. Even in terms of profit and reduction in damage, pheromones often compare favorably to the use of insecticides. In the case of treating cabbage against diamondback moth infestation, pheromone-based integrated pest management was found to be inexpensive ($62 relative to $123 per ha) resulting in a higher gross profit (ca $800 compared to $456 per ha) in comparison to the conventional practice with insecticides. The global market for pheromone-based control products is currently estimated to be approximately $200 million.

In 2010, around 40% of the sugarcane area in Brazil was treated by biological control, reducing pest damage by mass release of the larval parasitoid Cotesia flavipes (Hymenoptera: Braconidae) and the egg parasitoid Trichogramma galloi (Hymenoptera: Trichogrammatidae). The use of sex pheromones for monitoring or mating disruption has been successfully employed for other lepidopteran pests, though not yet for D. saccharalis, as field tests with the major sex pheromone component has shown low attractiveness to males compared to conspecific females.

The major female sex pheromone component of D. saccharalis has been identified as (9Z,11E)-hexadecadienal (Z9,E11-16:Ald), claimed in the U.S. patent application concerning the chemical synthesis of this compound to have been first reported by Hammond et al. at the meeting of Entomological Society of America in 1980. The first scientific publication identifying this pheromone component was in 2001 by Svatos̆ et al. In addition, three minor identified components, hexadecanal (16:Ald), (9Z)-hexadecenal (Z9-16:Ald) and (11Z)-hexadecenal (Z11-16:Ald), have been shown to elicit male antennal response and the more complex blends have been found to improve attraction of males in wind tunnel assays.

Studying how sex pheromones are biosynthesised by the insects is critical for development of biotechnological pheromone production to obtain the active compounds needed for pest management, and the successful use of insect enzymes for production of moth pheromones in yeasts and plant have been demonstrated. Moth sex pheromone biosynthesis involves genes in the large multigene families of fatty acyl desaturases (desaturases) and fatty acyl reductases (reductases), fatty alcohol oxidases and acetyltransferases. The biosynthesis of conjugated lepidopteran sex pheromones similar to Z9, E11-16:Ald has been described in moths from several families. The main pheromone components of Lampronia capitella (Prodoxidae) (9Z,11Z)-tetradecadienol (Z9,Z11-14:OH), Epiphyas postvittana (Tortricidae) (9E,11E)-tetradecadienyl acetate (E9,E11-14:OAc), and Spodoptera litura (Noctuidae) (9Z,11E)-tetradecadienyl acetate (Z9,E11-14:OAc) are made by one desaturase belonging to the clade of specific lepidopteran Δ11-desaturases, making both double bonds in sequence with a chain-shortening step in between. The major pheromone compounds of Cydia pomonella (Tortricidae) (8E,10E)-dodecadienol (E8,E10-12:OH) and Bombyx mori (Bombycidae) (10E,12Z)-hexadecadienol (E10,Z12-16:OH) are made by bifunctional desaturases that first introduce one double bond in the intermediate position and then turn this into the conjugated diene pheromone component. Two desaturases and several chain-shortening steps are involved in the biosynthesis of a pheromone component of Dendrolimus punctatus (Lasiocampidae), (5Z,7Z)-dodecadienol (Z5,Z7-12:OH), either using a desaturase homologous to common Δ9 acyl-CoA desaturases and a Lepidoptera specific Δ11-desaturase, or two Δ11-desaturases. The biosynthesis of the (7E,9Z)-dodecadienyl acetate (E7,Z9-12:OAc) pheromone component of Lobesia botrana (Tortricidae) involves a Δ11-desaturase and chain-shortening followed by the action of an elusive desaturase that makes a Δ7 double bond in the Z9-12:acyl. The biosynthetic pathways of these diunsaturated sex pheromone components may serve as hypotheses for D. saccharalis biosynthesis of Z9,E11-16:Ald.

As such, there is a need for improved methods with increased production of D. saccharalis pheromones and their precursors in plant or microbe factories.

### BRIEF SUMMARY

The present invention demonstrates the feasibility of the production of large amounts of insect (moth) pheromone precursors of sugarcane borer DIATRAEA SACCHARALIS (Fabricius) (Lepidoptera: Crambidae).

In accordance with one aspect, the invention relates to relates to a genetically modified plant having incorporated into its genome a heterologous gene(s) encoding a first fatty-acyl desaturase, a second fatty-acyl desaturase, and a fatty-acyl reductase, wherein the plant produces at least one Diatraea saccharalis pheromone precursor.

In accordance with another aspect, the invention relates to a genetically modified microorganism having incorporated into its genome a heterologous gene(s) encoding a first fatty-acyl desaturase, a second fatty-acyl desaturase, and a fatty-acyl reductase, wherein the microorganism produces at least one Diatraea saccharalis pheromone precursor.

In accordance with yet another aspect, the invention relates to a method of producing Diatraea saccharalis pheromone precursors. The method involves selecting a plant or a microorganism to be genetically modified, incorporating into its genome, a heterologous gene encoding a first fatty-acyl desaturase, a second fatty-acyl desaturase, and a fatty-acyl reductase to obtain a genetically modified plant or a genetically modified microorganism, and producing Diatraea saccharalis pheromone precursors from the genetically modified plant or the genetically modified microorganism.

By way of this invention, it is for the first time that it has been made possible to produce Diatraea saccharalis pheromone precursors and therefrom Diatraea saccharalis pheromones.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates a chromatogram of GC/MS analysis of total fatty acid composition from female Diatraea saccharalis pheromone gland;

FIG. 2 illustrates chromatograms of GC/MS selected ion monitoring (SIM) analyses of aldehydes from female Diatraea saccharalis pheromone gland extracts, showing incorporation from deuterated fatty acid precursors;

FIG. 3 illustrates female and male tissue transcript expression levels of Diatraea saccharalis putative desaturase, reductase and fatty alcohol oxidase genes involved in sex pheromone biosynthesis, identified from the transcriptome assembly;

FIG. 4 illustrates a phylogeny of Diatraea saccharalis full-length first desaturase and reductase sequences identified in transcriptome analysis, in relation to select sequences from other lepidopteran species;

FIG. 5 illustrates functional characterization of Diatraea saccharalis candidate desaturases and reductase in a yeast expression system;

FIG. 6 illustrates a pathway for biosynthesis of Diatraea saccharalis pheromone component fatty alcohol precursors;

FIG. 7 illustrates a synthesis of [16,16,16,15,15-2H5]-trans-11-hexadecenoic acid (D5-E11-16:acid); and

FIG. 8 illustrates ExN50 statistics for the transcriptome assembly.

### DETAILED DESCRIPTION

The detailed description set forth below is intended as a description of the presently preferred embodiment of the invention and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

### Definitions

In the context of the present application and invention, the following definitions apply:

As used herein, the terms “microbial,” “microbial organism,” and “microorganism” include any organism that exists as a microscopic cell that is included within the domains of archaea, bacteria or eukarya, the latter including yeast and filamentous fungi, protozoa, algae, or higher Protista. Therefore, the term is intended to encompass prokaryotic or eukaryotic cells or organisms having a microscopic size and includes bacteria, archaea, and eubacteria of all species as well as eukaryotic microorganisms such as yeast and fungi. Also, included are cell cultures of any species that can be cultured for the production of a chemical.

The term “genetic modification” implies the introduction of homologous and/or heterologous foreign nucleic acid molecules into the genome of a plant cell or into the genome of a microorganism, wherein said introduction of these molecules leads to an accumulation of insect pheromone precursors.

The term “recombinant microorganism” and “genetically modified microorganism” are used interchangeably herein and refer to microorganisms that have been genetically modified to express or to overexpress endogenous enzymes, to express heterologous enzymes, such as those included in a vector, in an integration construct, or which have an alteration in expression of an endogenous gene.

The term “expression” with respect to a gene sequence refers to transcription of the gene and, as appropriate, translation of the resulting mRNA transcript to a protein.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA.

The term “enzyme” as used herein refers to any substance that catalyzes or promotes one or more chemical or biochemical reactions, which usually includes enzymes totally or partially composed of a polypeptide or polypeptides but can include enzymes composed of a different molecule including polynucleotides.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

The term “fatty acid” as used herein refers to a compound of structure R—COOH, wherein R is a C₆ to C₂₄ saturated, unsaturated, linear, branched, or cyclic hydrocarbon and the carboxyl group is at position 1.

The term “fatty alcohol” as used herein refers to an aliphatic alcohol having the formula R—OH, wherein R is a C₆ to C₂₄ saturated, unsaturated, linear, branched, or cyclic hydrocarbon.

The term “fatty acyl-CoA” refers to a compound having the structure R—(CO)—S—R₁, wherein R₁ is Coenzyme A.

### Plant Platforms for Pheromone Production

In this disclosure, two plant platforms were utilized: Nicotiana benthamiana and Camelina sativa for pheromone production.

N. benthamiana is a close relative of N. tabacum, the most commonly grown commercial plant in the Nicotiana genus for its leaves to produce tobacco. Mature plants usually show a large variation in height, ranging from as tall as 1.5 meters to shorter than 200 mm. The Nicotiana species is favorable to work with in metabolic engineering aiming at production of pheromone compounds as they have relatively short production times, large area of leaves to output volatiles and are relatively easier to grow in controlled growth conditions. In addition, there is less concern about contaminating food supplies as they are not food crops.

Camelina was chosen as the oilseed production platform because it has limited use as a food crop and is considered an ideal system for rapid introduction and evaluation of fatty acid and other oil-related traits. Further, transgenes can easily be introduced into Camelina using a simple Agrobacterium-based method, and it has a relatively short life cycle that allows up to three generations in a year for evaluation of engineered traits. Camelina is also closely related to Arabidopsis thaliana, with a wealth of transgenic and genomic data for optimizing endogenous biosynthetic pathways for production of desired oil traits in seeds that typically are 30% to 40% oil by weight.

### Biosynthesis of Pheromones Using a Genetically Modified Plant

As discussed above, in a first aspect, the present invention relates to a genetically modified plant having incorporated into its genome a heterologous gene(s) encoding a first fatty-acyl desaturase, a second fatty-acyl desaturase, and a fatty-acyl reductase, wherein the plant produces at least one Diatraea saccharalis pheromone precursor. In an embodiment, the first fatty-acyl desaturase is a Δ9 desaturase and the second fatty-acyl desaturase is a Δ11 desaturase.

An exogenous fatty acyl desaturase described herein can be selected to catalyze the desaturation at a desired position on the hydrocarbon chain. Accordingly, in some embodiments, a Δ9 desaturase is capable of generating a double bond at C₉ position and Δ11 desaturase at C₁₁ position in the fatty acid or its derivatives, such as, for example, fatty acid CoA esters.

The major female sex pheromone component of D. saccharalis has been identified as (9Z,11E)-hexadecadienal (Z9,E11-16:Ald), whereas the three minor components have been identified as hexadecanal (16:Ald), (9Z)-hexadecenal (Z9-16:Ald), and (11Z)-hexadecenal (Z11-16:Ald).

The present invention explores the production of pheromone precursors in their fatty alcoholic form which on oxidation can be readily converted to the aldehydes which are the pheromones.

In one embodiment, the first fatty-acyl desaturase and the second fatty-acyl desaturase together catalyze the conversion of a C₁₆ fatty-acyl-CoA to at least one mono- or di-unsaturated C₁₆ fatty-acyl-CoA.

In one embodiment, the first fatty-acyl desaturase and the second fatty-acyl desaturase generate a double bond at the C₉ position and C₁₁ position, respectively. In an exemplary embodiment, the first fatty-acyl desaturase is Dsac_KPSE (SEQ ID NO: 4), a desaturase obtained from D. saccharalis. In another exemplary embodiment, the second fatty-acyl desaturase is selected from Dsac_NPTQ (SEQ ID NO: 1), and Dsac_NPAQ, both desaturases obtained from D. saccharalis. In certain embodiments, the Dsac_NPAQ can be that from the published genome (Dsac_NPAQ genome), or can be the Dsac_NPAQ-end (SEQ ID NO: 2) or Dsac_NPAQ-start (SEQ ID NO: 3) disclosed herein, any of which can be referred to herein as Dsac_NPAQ.

In one embodiment, the fatty-acyl reductase catalyzes the conversion of the at least one mono- or di-unsaturated C₁₆ fatty-acyl-CoA to at least one saturated, mono-, or di-unsaturated C₁₆ fatty alcohol. In an exemplary embodiment, the fatty-acyl reductase is selected from Dsac_FAR_3781 (SEQ ID NO: 15).

The at least one saturated, mono- or di-unsaturated C₁₆ fatty alcohol can be further oxidized to at least one saturated, mono-, or di-unsaturated C₁₆ fatty aldehyde. Biosynthesis of pheromones using a genetically modified microorganism

In a second aspect, the present invention relates to a genetically modified microorganism having incorporated into its genome a heterologous gene(s) encoding a first fatty-acyl desaturase, a second fatty-acyl desaturase, and a fatty-acyl reductase, wherein the microorganism produces at least one Diatraea saccharalis pheromone precursor. In an embodiment, the first fatty-acyl desaturase is a Δ9 desaturase and the second fatty-acyl desaturase is a Δ11 desaturase.

In one embodiment, the first fatty-acyl desaturase and the second fatty-acyl desaturase together catalyze the conversion of a C₁₆ fatty-acyl-CoA to at least one mono- or di-unsaturated C₁₆ fatty-acyl-CoA.

In one embodiment, the first fatty-acyl desaturase and the second fatty-acyl desaturase generate a double bond at the C₉ position and C₁₁ position, respectively. In an exemplary embodiment, the first fatty-acyl desaturase is SEQ ID NO: 4, a desaturase obtained from D. saccharalis. In another exemplary embodiment, the second fatty-acyl desaturase is selected from SEQ ID NO: 1, and Dsac_NPAQ, both desaturases obtained from D. saccharalis.

In one embodiment, the fatty-acyl reductase catalyzes the conversion of the at least one mono- or di-unsaturated C₁₆ fatty-acyl-CoA to at least one saturated, mono-, or di-unsaturated C₁₆ fatty alcohol. In an exemplary embodiment, the fatty-acyl reductase is selected as SEQ ID NO: 15.

The at least one saturated, mono- or di-unsaturated C₁₆ fatty alcohol can be further oxidized to at least one saturated, mono-, or di-unsaturated C₁₆ fatty aldehyde.

In an embodiment, the microorganism is a yeast. In an exemplary embodiment, the yeast is Saccharomyces cerevisiae.

### Method of Biosynthesis of Pheromones Using a Genetically Modified Plant or Microorganism

In a third aspect, the present invention relates to a method of producing Diatraea saccharalis pheromone precursors. The method involves selecting a plant or a microorganism to be genetically modified, incorporating into its genome, a heterologous gene encoding a first fatty-acyl desaturase, a second fatty-acyl desaturase, and a fatty-acyl reductase to obtain a genetically modified plant or a genetically modified microorganism, and producing Diatraea saccharalis pheromone precursors from the genetically modified plant or the genetically modified microorganism.

In an embodiment, the method involves catalyzing, by the first and the second fatty-acyl desaturases, conversion of a C₁₆ fatty-acyl-CoA to at least one mono- or di-unsaturated C₁₆ fatty-acyl-CoA. In another embodiment, the method involves catalyzing, by the fatty-acyl reductase, conversion of at least one mono- or di-unsaturated C₁₆ fatty-acyl-CoA into at least one Diatraea saccharalis pheromone precursor. In yet another embodiment, the method further involves oxidizing the at least one Diatraea saccharalis pheromone precursor to at least one Diatraea saccharalis pheromone.

### Pheromones

In different conditions, pheromones made using the invention's techniques and compositions including the pheromones can be employed to regulate Diatraea saccharalis insect behaviour and/or development. For instance, the pheromones can be employed to draw male Diatraea saccharalis insects to or away from a certain target region. Pheromones can be employed to draw Diatraea saccharalis insects away from agricultural regions that are particularly vulnerable. Insect monitoring, mass capturing, lure/attract-and-kill, or mating disruption strategies can all be utilised with the pheromones to draw in insects.

### Lures

In accordance with the embodiments of the present invention, lures may be coated with, sprayed with, or otherwise impregnated with one of the pheromone compositions described in the current disclosure.

### Traps

The pheromone compositions described in the disclosure may be employed in traps that are often used to draw Diatraea saccharalis insects. Such traps are widely utilised in many states and nations in pest eradication projects and are well recognised to those competent in the field. For retaining the pheromone mixture, the trap in one embodiment has one or more septa, containers, or storage receptacles. Thus, the current disclosure offers a trap that is loaded with at least one pheromone compound in certain embodiments. The pheromone compositions of the current disclosure can therefore be utilised in traps, for example, to entice Diatraea saccharalis insects as part of an approach for insect monitoring, mass trapping, mating disruption, or lure/attract and kill, for example, by incorporating a toxic substance into the trap to kill Diatraea saccharalis insects caught.

### Mating Disruption

Pheromones made using the disclosed techniques can also be utilised to interfere with mating. Introducing artificial stimuli (such as the pheromone composition given here) that confuses the insects and disturbs mating location and/or courting prevents mating and stops the reproductive cycle. This approach is known as mating disruption and is used to control insect infestations.

### Attract and Kill

The attract and kill approach, which can have the same results as mass-trapping, uses an attractant, such as a sex pheromone, to entice insects of the target species to an insecticidal chemical, surface, gadget, etc. for mass death and ultimate population reduction. When a synthetic female sex pheromone is used to attract male pests, such as moths, in an attract-and-kill technique, for example, a significant number of male moths must be killed over a prolonged period of time in order to restrict mating and reproduction and ultimately control the pest population.

In the following section, the aspect is described by way of examples to illustrate the processes of the invention. However, these do not limit the scope of the present invention. Several variants of these examples would be evident to persons ordinarily skilled in the art.

### Example

### Materials and Methods

### Reference and Deuterated Chemicals

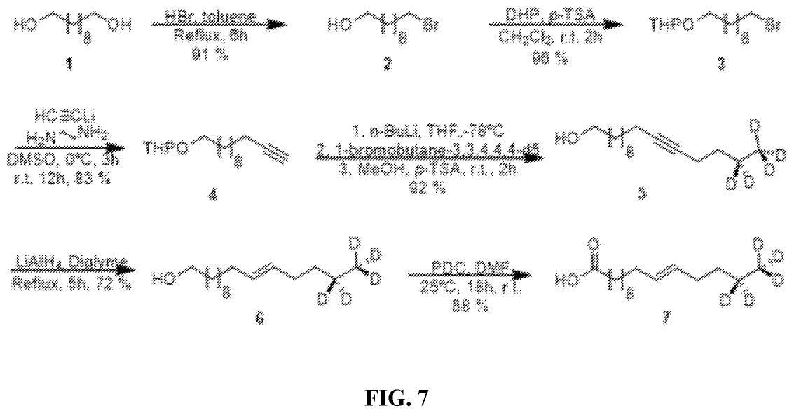

Reference chemicals for identification of pheromone gland (PG) compounds were purchased from Pherobank (Wijk bij Duurstede, The Netherlands), including hexadecanal (16:Ald), four isomers of hexadecenal (E/Z9-16:Ald and E/Z11-16:Ald) and the four isomers of (9,11)-hexadecadienal (Z9,E11-16:Ald). The aldehydes were used to prepare corresponding acids, methyl esters and alcohols according to previously reported protocols (Bjostad and Roelofs 1984; Corey and Schmidt 1979; Corso et al. 1998). Deuterium-labelled fatty acid for in vivo labelling, [16,16,16-²H₃]-hexadecanoic acid (D₃-16:acid), was purchased from Larodan (Malmo, Sweden), [16,16,16,15,15,14,14,13,13-²H₉]-cis-11-hexadecenoic acid (D₉-Z11-16:acid) and [8,8,7,7,6,6,5,5,4,4,3,3,2,2-²H₁₄]-cis-9-hexadecenoic acid (D₁₄-Z9-16:acid) from Cayman Chemicals (MI, USA). [16,16,16,15,15-²H₅]-trans-11-hexadecenoic acid (D₅-E11-16:acid) was prepared following Zarbin et al. (2007) (FIG. 7). 1,10-decanediol was used as starting material and subjected to monobromination with hydrobromic acid (48%) and toluene under reflux for 6 h to synthesize 10-bromodecan-1-ol. The hydroxyl group was protected with 3,4-dihydro-2H-pyran in dichloromethane, with p-toluenesulfonic acid (p-TSA) as catalyst at room temperature for 2 h to give 2-((10-bromodecyl)oxy)tetrahydro-2H-pyran. The alkyne 2-(dodec-11-yn-1-yloxy)tetrahydro-2H-pyran was generated coupling the bromide with lithium acetylide (ethylene diamine complexed) using dimethyl sulfoxide as solvent. The mixture was stirred for 3 h at 0° C., then for another 12 h at room temperature. This product was converted into the anion with n-butyllithium in tetrahydrofuran at −78° C. and the mixture was warmed to 0° C. [4,4,4,3,3-²H₅]-1-bromobutane (Qmx Laboratories Ltd, Dunmow, UK) was added after 30 min and the reaction was stirred overnight. The crude product was directly deprotected by p-TSA in methanol at room temperature for 2 h, producing [16,16,16,15,15-²H₅]-hexadec-11-yn-1-ol. [16,16,16,15,15-²H₅]-trans-11-hexadecanol was obtained by reaction with lithium aluminium hydride in diglyme under reflux for 5 h. The oxidation of the alcohol with pyridinium dichromate in dimethylformamide for 18 h at room temperature produced D₅-E11-16:acid at 88% yield. All materials were purchased from Merck unless otherwise stated.

### Insects, Dissection, and Extraction of Sex Pheromone Glands

Insects were procured from the Entomology Department of the Superior School of Agriculture Luiz de Queiroz at University of Sao Paulo, Brazil and reared in the lab on an artificial diet of soy flour, sugar and wheat germ, at conditions of 23° C., 70% relative humidity and a light:dark cycle of 16:8 h. Males and females were kept separately after the pupal stage. The time of dissection and number of sex pheromone glands (PGs) extracted for pheromone and total lipid/precursor analysis was based on pheromone amounts observed in the study by Batista-Pereira et al. (2002). Pheromone glands of 1 to 3 day old virgin females were dissected 3-5 h into scotophase and extracted in 15 μL heptane (Merck) per 5 glands. The solvent was transferred to a new vial after 15 min for pheromone analysis by GC/MS. For subsequent total lipid extraction and precursor GC/MS analysis the same sample was extracted again with chloroform:methanol (2:1 v/v) (Merck), overnight at room temperature, the solvent evaporated by a gentle stream of N₂, and subjected to base methanolysis (Bjostad and Roelofs 1984) for transformation of lipids into fatty acid methyl esters (FAMEs). Identification of double-bond position of monounsaturated compounds was done using DMDS-adducts of FAMEs (Buser et al. 1983), produced by using 100 μL dimethyl disulfide (Merck) and 20 μL 5% I₂ in diethyl ether (Merck), incubating at 40° C. overnight, then mixing with 50 μL 5% aqueous sodium thiosulfate (Merck) and 50 μL heptane.

### Gas Chromatography/Mass Spectrometry Analysis of Sex Pheromone Components and Fatty Acid Precursors

Pheromone- and FAME PG extracts were analysed in split less mode using an Agilent 5975 mass detector (Agilent Technologies, Palo Alto, Calif., USA) coupled to an Agilent 6890 series gas chromatograph or an Agilent 5977B mass detector coupled to an Agilent 8890 series gas chromatograph, both fitted with an HP-INNOWax column (30 m×0.25 mm i.d., 0.25 m film thickness; J & W Scientific, Agilent Technologies, Santa Clara, Calif., USA). The GC inlet was set to 250° C. and the oven program to 80° C. for 1 min, increase of 10° C./min to 230° C., held for 10 min. The temperature of the transfer line was 280° C. and the MS source 230° C. DMDS-adducts were analysed using an Agilent 5975C mass detector coupled to an Agilent 7890A series gas chromatograph fitted with a HP-5MS column (30 m×0.25 mm i.d., 0.25 m film thickness, J & W Scientific, Agilent Technologies, Santa Clara, Calif., USA). The GC inlet was set to 260° C. and the oven program to 80° C. for 2 min, increase of 15° C./min to 140° C., then 5° C./min to 260° C. held for 15 min. The temperature of the transfer line was 280° C. and the MS source 230° C. Helium was used as the carrier gas.

### In Vivo Labelling

Deuterium-labelled fatty acids D₃-16:acid, D₉-Z11-16:acid, D₅-E11-16:acid, D₁₄-Z9-16:acid and DMSO as a control were used for in vivo labelling, to monitor incorporation into the pheromone components and the potential pheromone precursors in the pheromone biosynthetic pathway. Labelled compound, 16 μg in a volume of 0.4 μL DMSO, was topically applied to the extruded female pheromone gland and abdominal tip 1 h before extraction of the pheromone gland. Pheromone gland extraction and pheromone and precursor analyses by GC/MS were performed as described above.

### Sequencing and Transcriptome and Phylogenetic Analyses

Thirty female PGs and 32 male abdominal tips for transcriptome analysis were dissected as described above and stored immediately at −80° C. prior to RNA extraction. RNA extraction was done using TRIzol reagent (Thermo Fisher) and RNA cleanup and concentration using the RNeasy Micro kit (QIAGEN), both steps following the manufacturers' instructions. The RNA concentration was measured using a 2100 Bioanalyzer system (Agilent). Library preparation with Illumina TruSeq poly-A enrichment and 150 bp paired-end Illumina sequencing using a NovaSeq6000 system was done by SciLifeLab National Genomics Infrastructure (Stockholm, Sweden), for one female and one male replicate. FastQC v0.11.5 (bioinformatics.babraham.ac.uk/projects/fastqc) was used to assess quality of the reads, and low quality raw reads were filtered and adaptors removed using Trimmomatic v0.36 (Bolger et al. 2014) and Prinseq v0.20.4 (Schmieder and Edwards 2011). Assembly was done using the Trinity software package v2.8.2 (Grabherr et al. 2011; Haas et al. 2013) with default parameters except normalize_max_read_cov 50—min_kmer_cov 2—min_glue 2—KMER_SIZE 23, and completeness of the assembly assessed with BUSCO v3.0.2b and the Insecta- and Endopterygota datasets (https://busco.ezlab.org/). TransDecoder v5.0.1 (github.com/TransDecoder/) was used to extract ORFs and predict protein coding regions, and differential expression between female and male tissues estimated with RSEM v1.3.1 (Li and Dewey 2011) and Trinity package scripts align_and_estimate_abundance.pl and abundance_estimates_to_matrix.pl. Fatty acid desaturases were found using the FA_desaturase (PF00487) family of the Pfam domain database (Mistry et al. 2020) together with HMMER v3.2.1 (hmmer.org), and fatty acid reductases and fatty alcohol oxidases were found by BLAST (Altschul et al. 1990) homology search with other lepidopteran sequences. For phylogenetic analyses, D. saccharalis desaturases and reductase amino acid sequences, together with other lepidopteran sequences available from GenBank (ncbi.nlm.nih.gov), were aligned using MAFFT (Katoh et al. 2002) and scoring matrix BLOSUM62, and maximum-likelihood phylogenies were constructed with FastTree v7.450 (Katoh et al. 2002; Katoh and Standley 2013) and visualised with FigTree v1.4.4 (github.com/rambaut/figtree/).

### Cloning and Yeast Functional Assay of Insect Genes

cDNA was synthesised from the PG RNA sample with the Thermoscript RT-PCR kit (Thermo Fisher) following the manufacturer's protocol. To verify transcript sequences, all full-length gene ORFs were amplified and Sanger sequenced. For yeast episomal expression, desaturase—and reductases were cloned using Gateway technology (Thermo Fisher) into pDONR221 followed by pYEX-CHT (Patel et al. 2003) or pYES-DEST52 vectors, and transformed into the yeast strain Δole1/Δelo1 (MATa elo1::HIS3 ole1::LEU2 ade2 his3 leu2 ura3) (Schneiter et al. 2000) or INVSc1 (MATa his3D1 leu2 trp1-289 ura3-52 MAT his3D1 leu2 trp1-289 ura3-52) (Thermo Fisher) using the S.c. EasyComp transformation kit (Thermo Fisher) following the manufacturers' protocols. Uracil prototrophs were selected for and cultivated in medium containing 1.92 g/L dropout medium lacking uracil (Formedium), 6.7 g/L yeast nitrogen base (Merck), 0.08 g/L adenine (Merck), 1.5% tergitol (Merck), 100 mM oleic acid (Merck) and 2% glucose (Merck) or 2% galactose (Merck) and 1% raffinose (Merck). The medium also contained 1 mM 12:Me and 14:Me, except for expressions of desaturases SEQ ID NO: 1, Dsac_NPAQ and SEQ ID NO: 4. A 24-72 h pre-cultivation was followed by 48-72 h incubation at 30° C. of 4-50 mL cultures, where the heterologous gene expression was induced with a final concentration of 0.5-1 mM CuSO₄ (Merck) and/or 2% galactose. Cells were harvested and subjected to total lipid GC/MS analysis as described above, or as follows: extraction with 1 mL methanol:chloroform (2:1, v/v) and 1 mL 0.075 M acetic acid (Merck), followed by running the organic phase on a silica gel TLC plate (Silica gel 60, Merck) that was developed in heptane:DEE:HAc (85:15:1,v/v/v), the bands visualized by spraying water and target gel areas collected separately into 4 mL vials and extracted with 1 mL methanol:chloroform (2:1, v/v) in a sonication bath for 2 min. The extract was then centrifuged at 2,000 g for 1 min. The supernatant was transferred to a new vial and 1 mL 0.075 M acetic acid was added to partition the lipids into chloroform. The chloroform phase containing alcohols was transferred into a new vial and evaporated to dryness, followed by addition of 40 μL heptane and GC/MS analysis as described above. For yeast integrative expression, constructs were cloned by fusion PCR and Gateway technology into vector pCfB2875, modified from Maury et al. (2016), and transformed into INVSc1 and cultivated as described above, in 25-100 mL and for four days. Total broth or only supernatant was extracted using 2 mL heptane/25 mL culture and analysed by GC/MS as described above.

### Results

### Analysis of Sex Pheromone Gland Compositions

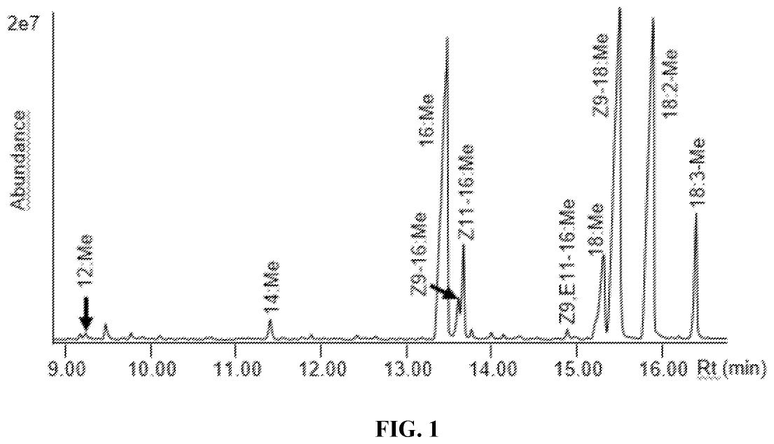

Extracts of pheromone glands (PGs) contained the previously identified sex pheromone components of D. saccharalis (16:Ald, Z9-16:Ald, Z11-16:Ald and Z9,E11-16:Ald) and their corresponding fatty acid precursors (FIG. 1), identified by their mass spectra and retention times relative to those of synthetic references, and by DMDS adduct analysis for the monounsaturated compounds. Extracts also contained the three other isomers of Δ9,Δ11-16:Ald/acid, but no E9-16:Ald/acid, E11-16:Ald/acid or other monounsaturated C₁₆ acid compounds were detected. The amount of Z9,E11-16:Ald was around 1 ng/PG, and the percent ratio between Z9,E11-16:Ald, Z11-16:Ald and 16:Ald was 66±9.9, 19±6.7 and 15±5.2, respectively. Z9-16:Ald amount was too low to be quantified. Using ion m/z 236, the percent ratio between Δ9,Δ11-16:Ald isomers Z,E, Z,Z and E,E was 87±2.6, 13±1.6 and 1±1.2, respectively, while isomer E,Z amount was too low to be quantified. In the fatty acid precursor sample, the percent ratio between Z9,E11-16:Me, Z11-16:Me, 16:Me and Z9-16:Me was 75±18, 19±13, 3±3 and 3±3, and using ion m/z 266, the percent ratio between Δ9,Δ11-16:Me isomers Z,E, E,Z, Z,Z and E,E was 73±10, 3±3, 11±2 and 13±10. The abundance of the E,E-isomer is probably a little bit overestimated because 18:Me also contributes to the m/z 266 area at the same retention time.

### In Vivo Labeling for Elucidation of Sex Pheromone Biosynthetic Pathway

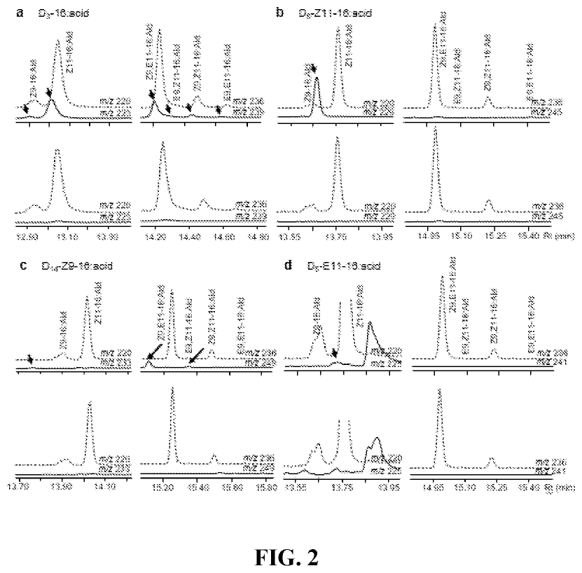

For elucidation of the pathway for sex pheromone biosynthesis, deuterium-labelled potential pheromone precursors were used in in vivo labelling experiments (FIG. 2). For D₃-16:acid, label-incorporation was detected in all four 9,11-16:Ald/acid isomers, Z9-16:Ald/acid, Z11-16:Ald/acid and 16:acid (FIG. 2a). For D₁₄-Z9-16:acid, label-incorporation was detected in Z9,E11-16:Ald/acid, Z9,Z11-16:Ald/acid and Z9-16:Ald/acid (FIG. 2b). For D₉-Z11-16:acid and D₅-E11-16:acid, the only detected incorporation was in Z11-16:Ald/acid and E11-16:Ald, respectively (FIGS. 2c and 2d).

### Transcriptome and Phylogenetic Analyses of Sex Pheromone Biosynthesis Genes

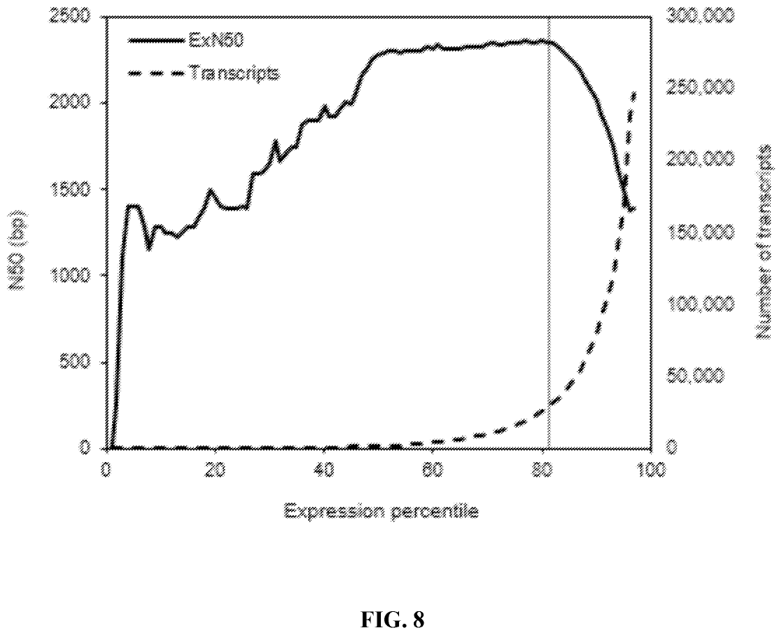

Transcriptome sequencing yielded 390 M raw reads per sample, and 282-302 M reads per sample after quality filtering. The assembly using both female and male samples resulted in 267,839 total assembled transcripts with an average length of 831 bp, N50 length of 1,689 bp and an overall alignment rate of 77%. Looking at ExN50 statistics, 25,333 transcripts are covered by 83% of reads and N50 length of this subset is 2,360 bp (FIG. 8). When compared to the 1,658 total BUSCO groups of the Insecta dataset, the completeness of the assembly was assessed to 94%, and 88% when compared to the 2,442 total BUSCO groups of the Endopterygota dataset.

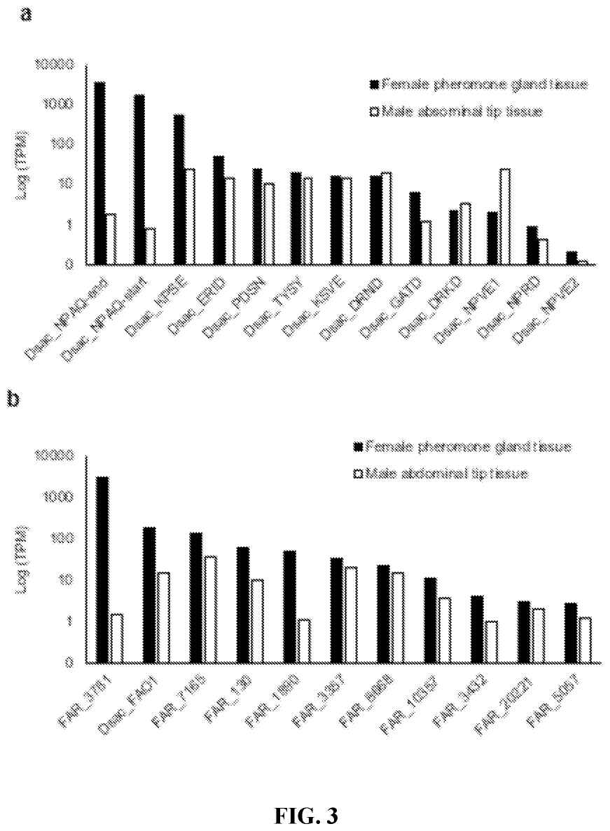

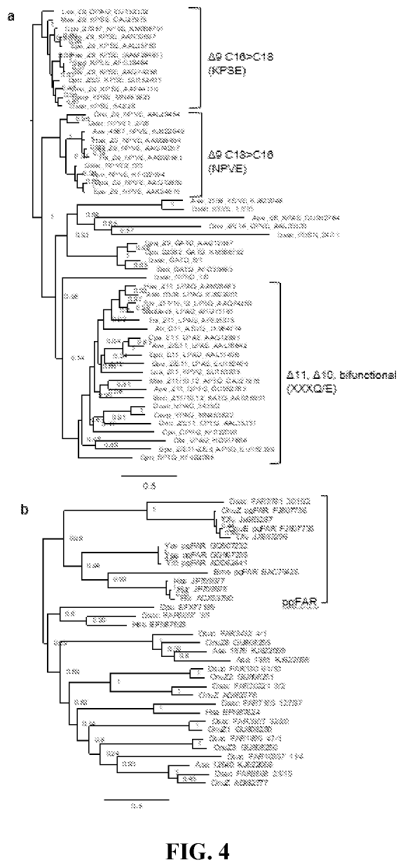

The transcriptome assembly was searched for putative desaturase, reductase and alcohol oxidase genes involved in sex pheromone biosynthesis, and 12, ten and one full-length genes, respectively, were found by homology search with known lepidopteran genes (Table 1 and transcript assembly nucleotide sequences). For four of these genes the expression level was considered both female-biased (expression ratio female:male >10) and relatively high (>100 TPM) (FIG. 3). Phylogenetic alignment of first desaturases with similar sequences from other lepidopteran species (excluding the more divergent sphingolipid desaturases and front-end desaturases (Cyt-b5-r) that were also identified) showed that SEQ ID NO: 4 clustered within the putative Δ9-desaturase clade and Dsac_NPAQ within the Δ11-desaturase clade (FIG. 4a). The Dsac_NPAQ consensus sequence was assembled from two overlapping transcripts, resulting in a protein sequence with ten ambiguous amino acid residues in the overlap. All full-length desaturase genes found the in the transcriptome assembly, except for Dsac_NPVE2, could be confirmed by cDNA amplification and sequencing of the ORF. An exact match to the transcriptome assembly sequence of Dsac_NPAQ could not be amplified from cDNA, instead the variant Dsac_NPTQ (SEQ ID NO: 1) was amplified, different in seven amino acids residues compared to the ambiguous Dsac_NPAQ transcript and 33 amino acid residues compared to the Dsac_NPAQ (genome) sequence. SEQ ID NO: 15 clustered within the pgFAR clade of known lepidopteran reductases (FIG. 4b).

TABLE 1
Expression level of full-length putative desaturase, reductase and fatty alcohol oxidase
genes involved in sex pheromone biosynthesis, identified from the transcriptome assembly.

| | | | Expression | Expression | | |
|---|---|---|---|---|---|---|
| | | | level female | level | | |
| | | | pheromone | male | Ratio | Functional |
| | Assembly | Length | gland tissue | abdominal tip | female:male | assay results |
| Desaturases | transcript ID | (aa) | (TPM) | tissue (TPM) | expression | in this study |
| Dsac_NPAQ-end | DN21815_c0_g2_i2 | 249 | 3435.0 | 1.8 | 1951.7 | Yeast |
| (SEQ ID NO: 2) | | | | | | |
| Dsac_NPAQ-start | DN1252_c4_g1_i2 | 230 | 1666.0 | 0.8 | 2135.9 | Yeast |
| (SEQ ID NO: 3) | | | | | | |
| Dsac_KPSE (SEQ | DN1192_c1_g1_i3 | 353 | 542.3 | 25.8 | 21.0 | Yeast |
| ID NO: 4) | | | | | | |
| Dsac_ERID (SEQ | DN156_c0_g1_i9 | 457 | 51.2 | 14.8 | 3.5 | Yeast |
| ID NO: 5) | | | | | | |
| Dsac_PDSN (SEQ | DN715_c0_g1_i2 | 325 | 24.2 | 11.1 | 2.2 | Yeast |
| ID NO: 6) | | | | | | |
| Dsac_TYSY (SEQ | DN4810_c2_g3_i1 | 322 | 19.4 | 15.0 | 1.3 | Yeast |
| ID NO: 7) | | | | | | |
| Dsac_KSVE (SEQ | DN9980_c4_g1_i1 | 376 | 16.6 | 14.5 | 1.1 | Yeast |
| ID NO: 8) | | | | | | |
| Dsac_DRND (SEQ | DN2204_c1_g2_i2 | 453 | 16.2 | 19.1 | 0.8 | Yeast |
| ID NO: 9) | | | | | | |
| Dsac_GATD (SEQ | DN41058_c0_g1_i1 | 374 | 6.3 | 1.2 | 5.4 | Yeast |
| ID NO: 10) | | | | | | |
| Dsac_DRKD (SEQ | DN8475_c1_g1_i2 | 451 | 2.2 | 3.5 | 0.6 | Yeast |
| ID NO: 11) | | | | | | |
| Dsac_NPVE1 | DN3246_c0_g1_i6 | 352 | 2.0 | 25.6 | 0.1 | Yeast |
| (SEQ ID NO: 12) | | | | | | |
| Dsac_NPRD (SEQ | DN50866_c0_g1_i3 | 359 | 0.9 | 0.4 | 1.9 | Yeast |
| ID NO: 13) | | | | | | |
| Dsac_NPVE2 | DN3281_c0_g1_i1 | 353 | 0.2 | 0.1 | 1.5 | |
| (SEQ ID NO: 14) | | | | | | |

Reductases

| | | | | | | |
|---|---|---|---|---|---|---|
| Dsac_FAR_3781 | DN3781_c0_g1_i12 | 474 | 3098.7 | 1.6 | 1986.4 | Yeast |
| (SEQ ID NO: 15) | DN7165_c1_g1_i1 | 511 | 130.7 | 36.1 | 3.6 | |
| Dsac_FAR_7165 | | | | | | |
| (SEQ ID NO: 16) | DN130_c0_g1_i26 | 626 | 62.2 | 10.1 | 6.2 | |
| Dsac_FAR_130 | | | | | | |
| (SEQ ID NO: 17) | DN1890_c1_g2_i4 | 526 | 48.4 | 1.2 | 42.1 | |
| Dsac _FAR_1890 | | | | | | |
| (SEQ ID NO: 18) | DN3357_c0_g1_i5 | 517 | 32.6 | 19.7 | 1.7 | |
| Dsac_FAR_3357 | | | | | | |
| (SEQ ID NO: 19) | DN8668_c1_g1_i8 | 499 | 21.4 | 14.6 | 1.5 | |
| Dsac_FAR_8668 | | | | | | |
| (SEQ ID NO: 20) | DN10357_c2_g2_i1 | 522 | 11.2 | 3.5 | 3.2 | |
| Dsac_FAR_10357 | | | | | | |
| (SEQ ID NO: 21) | DN3432_c0_g1_i12 | 436 | 3.9 | 1.0 | 3.9 | |
| Dsac_FAR_3432 | | | | | | |
| (SEQ ID NO: 22) | DN20221_c0_g1_i1 | 550 | 3.0 | 1.9 | 1.6 | |
| Dsac_FAR_20221 | | | | | | |
| (SEQ ID NO: 23) | DN5057_c0_g2_i1 | 523 | 2.8 | 1.3 | 2.2 | |
| Dsac_FAR_5057 | | | | | | |
| (SEQ ID NO: 24) | | | | | | |

Fatty alcohol oxidases

| | | | | | |
|---|---|---|---|---|---|
| Dsac_FAO1 | DN4342_c3_g2_i3 | 623 | 173.4 | 14.2 | 12.2 |
| (SEQ ID NO: 25) | | | | | |

### Yeast Functional Assays of Desaturases and Reductase

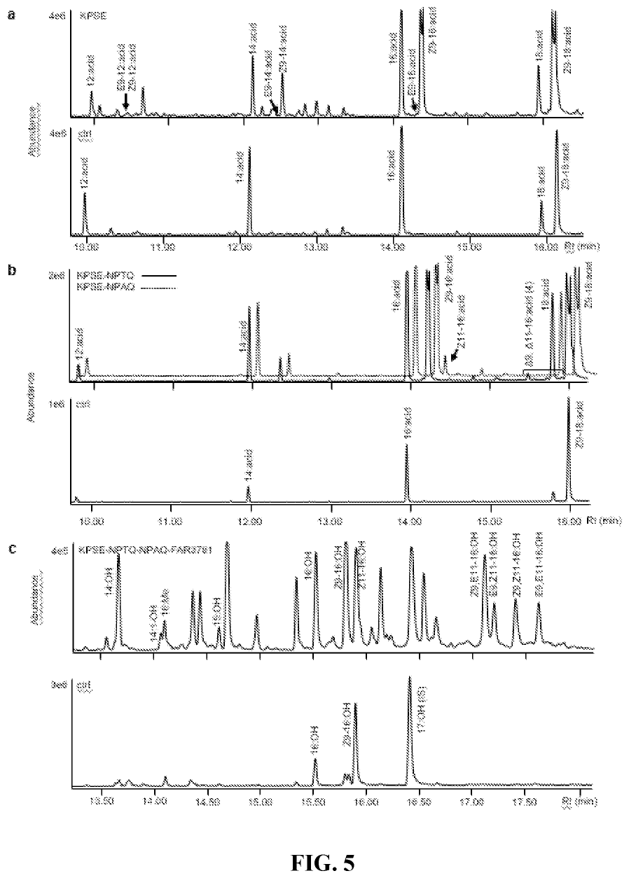

Desaturases and reductases for which expression in the transcriptome analysis was considered both female-biased (female:male >10) and relatively high (>100 TPM), and all remaining full-length desaturases found in the transcriptome, were together with the desaturase Dsac_NPAQ from the published genome (Borges dos Santos et al. 2020), functionally characterised in a yeast expression system. After expression, their products were methylated and analysed by GC/MS (FIG. 5). All mono- and diunsaturated fatty acids in the form of corresponding methyl esters (FAMEs) were identified by their mass spectra and retention times relative to those of synthetic reference compounds. The putative Δ9-desaturase SEQ ID NO: 4 expressed in a Δole1/Δelo1 yeast background (knock-out of native yeast Δ9 desaturase and fatty acid elongase, and supplied with oleic acid (OA) during cultivation, produced (Z9)-hexadecenoic acid (Z9-16:acid) as the most abundant acid in the cells (FIG. 5a). Other monounsaturated acids produced by SEQ ID NO: 4 were all isomers of Δ9-dodecenoic acid (E/Z9-12:acid) and tetradecenoic acid (E/Z9-14:acid), and (E9)-hexadecenoic acid (E9-16:acid). No (E9)-octadecenoic acid (E9-18:acid) was detected (DMDS analysis). No monounsaturated acids were produced in the empty vector control cells. The putative Δ11-desaturases SEQ ID NO: 1 and Dsac_NPAQ (genome) were expressed separately, together with SEQ ID NO: 4, in the Δole1/Δelo1 yeast background, and both strains produced Z9-16:acid, Z11-16:acid and all four isomers of (9,11)-hexadecadienoic acid (Δ9,Δ11-16:acid), none of which were found in the empty vector control cells (FIG. 5b). The NPTQ strain produced only trace amounts of Z11-16:acid and the NPAQ strain only trace amounts of the Δ9,Δ11-16:Me isomers. No E11-16:acid was detected. Expression of these two Δ11-desaturases without SEQ ID NO: 4 resulted in production of Z11-16:acid only (chromatogram not shown). A WT yeast background with abundant native Z9-16:acid was used to simultaneously express the four genes SEQ ID NO: 4, SEQ ID NO: 1, Dsac_NPAQ (genome) and SEQ ID NO: 15 (FIG. 5c), and produced Z9,E11-16:OH at an average titre of 2.7±0.4 mg/L in the broth of 25 mL-scale cultivations. The percent ratio between the four pheromone blend compound precursors Z9,E11-16:OH, Z11-16:OH, 16:OH and Z9-16:OH was 22±4.5, 45±6.2, 17±4.2 and 17±5.3, respectively. The percent ratio between the four Δ9, Δ11-16:OH isomers, Z,E, E,Z, Z,Z and E,E was 53±4.0, 11±1.4, 20±1.1 and 16±4.1, respectively. No difference was seen between the episomal and genome integrated expressions (chromatogram not shown). The remaining full-length desaturases expressed in the Δole1/Δelo1 yeast background, showed no desaturation activities compared to the control, except for Dsac_NPVE1 which produced Z9-14:acid and Z9-16:acid (chromatograms not shown).

### DISCUSSION

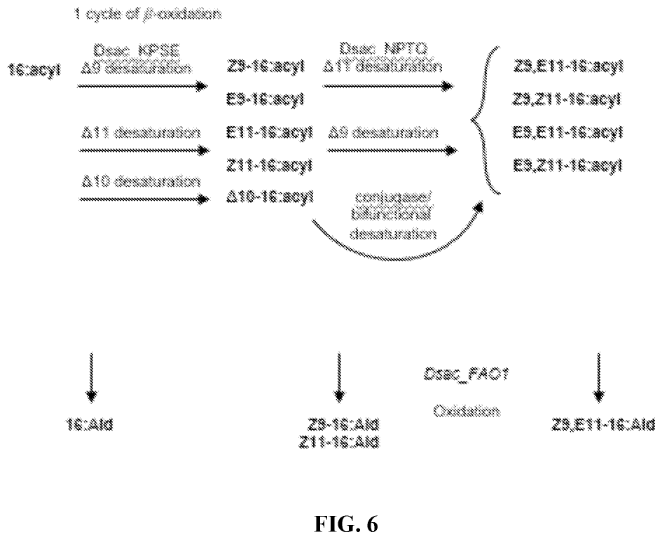

We have shown that biosynthesis of the major D. saccharalis sex pheromone component, Z9,E11-16:Ald, involves two different desaturases acting in sequence, the Δ9 desaturase SEQ ID NO: 4 acting on palmitic acid and producing Z9-16:acid, followed by the Δ11 desaturation by SEQ ID NO: 1 to produce Z9,E11-16:acid (FIG. 6). The reductase har turns this precursor into an alcohol. These results are significant for a prospective future biotechnological production of the D. saccharalis sex pheromone for use in pest management.

The presented pathway is supported by the presence of 16:acid and Z9-16:acid precursors in the PG while no E11-16:acid or E/Z10-16:acid was seen. This indicates that saturated 16:acid is the precursor being desaturated (as opposed to for example 18:acid), and that the Z9 double bond in the diene is made first, instead of the E11 double bond or the Z9,E11 double bond being made by a bifunctional desaturase from a Δ10 double bond. In vivo labelling showed that 16:acid and Z9-16:acid precursors were incorporated into the pheromone component whereas E11-16:acid was not. The functional assay of the desaturases in the yeast background deficient in native Δ9 desaturation activity showed that SEQ ID NO: 4 displays broad Δ9 desaturation activity with a preference for producing Z9-16:acid, the precursor of Z9,E11-16:Ald and also the minor pheromone component Z9-16:Ald. SEQ ID NO: 1 displays Δ11 desaturation activity to produce the major pheromone component precursor, and the other three isomers, Z9,Z11-16:acid, E9,E11-16:acid and E9,Z11-16:acid. It also produces the precursor of pheromone component Z11-16:acid. This type of desaturation pathway is similar to that seen for the pine caterpillar moth D. punctatus, where a Δ9 (Dpud9_KPSE) and a Δ11 (Dpud11_LPAE) desaturase are involved in the biosynthesis of a diene pheromone component precursor Z5,E7-12:acid (Liénard et al. 2010). Our in vivo labelling experiments could establish that Z9-16:acid is the precursor of two of the dienes isomers seen in the PG, Z9,E11-16:acid and Z9,Z11-16:acid, while no incorporation was seen into the two isomers with an E9 double bond. Possibly these come from the E9-16:acid precursor produced by SEQ ID NO: 4.

The pgFAR SEQ ID NO: 15 was in the yeast assay shown to have a broad specificity and ability to reduce all the D. saccharalis pheromone precursor components into their respective alcohols. This pgFAR and the two described desaturases being part of the pheromone biosynthesis pathway is corroborated by their high expression levels and female PG biased expressions. Even if mRNA expression level does not always have an obvious biological significance and is not an undisputable indicator of protein expression level (Koussounadis et al. 2015), transcript expression level and PG bias has in sex pheromone biosynthesis studies so far been a good indicator for involvement in the pathway (Antony et al. 2015; Strandh et al. 2008; Zhang et al. 2014). A less expressed gene candidate might be involved, but often this lower expression represents a suboptimal sampling time or less discriminate tissue dissection. A putative fatty alcohol oxidase (FAO) responsible for the last step in D. saccharalis pheromone biosynthesis, namely conversion of the alcohol precursors into their aldehyde pheromone counterparts, was identified in the transcriptome based on similarity to other putative moth FAOs (Xia et al. 2022), its high expression level and female PG bias. An assay for functional characterisation of a FAO or other aldehyde oxidases by heterologous expression in yeast and monitoring of aldehyde production has not yet been established and may be difficult due to the toxicity of aldehydes in such a system.

An unambiguous Dsac_NPAQ transcript could not be inferred from the de novo transcriptome assembly, and neither could a transcript identical to the one ultimately amplified from cDNA, SEQ ID NO: 1. The completeness of the assembly was assessed to be high at 94% when comparing to the BUSCO dataset of conserved Insecta genes. Ambiguities in transcriptome assemblies can be related to allele variation or gene families (paralogs), which could explain the difficulty observed in resolving this sequence that possibly correspond to two highly similar transcripts (Razo-Mendivil et al. 2020). Lepidopteran desaturases are part of a large gene family of both ancient and more recent duplicated and evolved genes (Lienard et al. 2008). The ambiguous sequence might be two very similar genes merged because of low stringency in assembly parameters, creating fragmented or chimeric sequences. Sometimes fragmented sequences can be completed by increasing the k-mer size in the assembly parameters, but highly similar transcripts resulting in chimeras (or lowly expressed paralogs, with low coverage) might possibly only be resolved by lowering the k-mer size, which in turn can result in a highly fragmented assembly (Gruenheit et al. 2012). If the consensus sequence for Dsac_NPAQ is chosen as the transcript assembly most similar to Dsac_NPAQ from the available D. saccharalis genome assembly (Borges dos Santos et al. 2020), it's 17 amino acid residues different from the SEQ ID NO: 1 sequence amplified from cDNA. It might not be possibly to resolve two such highly similar sequences with similar expression levels, by short-read transcriptome sequencing and de novo assembly. Two such sequences would be more easily resolved if one of them had lower expression, and then a certain coverage cut-off level could be used to unambiguously assemble only the sequence with high expression level (Gruenheit et al. 2012). The D. saccharalis genome assembly presented in Borges dos Santos et al. (2020) is 87% complete assessed by the BUSCO Insecta dataset, and does resolve two full-length genes, Dsac_NPAQ (genome) and SEQ ID NO: 1 (genome), differing in 35 amino acid residues and also in their intron sequences. These are highly similar to Dsac_NPAQ from this study's transcriptome assembly and SEQ ID NO: 1 amplified from the transcriptome cDNA, respectively. These could represent different alleles (Lassance et al. 2010), or a recent duplication event creating two paralogs that are still both functional and exhibit high expression levels, where one might evolve to become redundant in future pheromone biosynthesis of D. saccharalis. It could also be that both paralogs contribute to the specificity of the D. saccharalis pheromone blend. Both versions of this desaturase expressed in yeast in this study (SEQ ID NO: 1 (cDNA) and Dsac_NPAQ (genome)) were functionally capable of producing the pheromone component precursors Z11-16:acid and Z9,E11-16:acid (and its isomers), although at different ratios. These sequences differ in 33 amino acid residues, possibly revealing positions in the primary sequence that are functionally redundant, but also responsible for the ratio difference. 30 out of the 33 are considered conservative substitutions, and none are within domains characterised as important for the catalytic function, such as the conserved histidine-residues, the CoA-binding site or the coordinating carboxylates (Bai et al. 2015). 14 substitutions are within either trans-membrane domains or alpha-helices as characterised in the crystal structure of the mammalian stearoyl-CoA desaturase in Bai et al. (2015), and 19 are in domains not structurally or functionally defined.

The alcohol precursor titres presented in this study are too low to be immediately relevant for industrial biotechnological production and use in prospective IPM applications. Expression of SEQ ID NO: 4, 2× SEQ ID NO: 1/NPAQ and SEQ ID NO: 15 produced a titre of only 2.7±0.4 mg/L Z9,E11-16:OH precursor in the broth of small-scale cultivations. Before a large-scale yeast production of D. saccharalis pheromone precursor(s) can be initiated, using the identified biosynthetic pathway, it's relevant to optimise with regard to yeast host, platform strain and fermentation conditions, possibly even enzyme engineering for more active and specific desaturases. Some of these optimisations may involve manipulating the flux in the yeast fatty acid biosynthesis pathways, the copy number of pheromone biosynthesis genes and eliminating production of unwanted side-products (Holkenbrink et al. 2020). A biotechnological production could consist of heterologous synthesis of all the pheromone component precursors at a biologically relevant ratio, for trapping and monitoring purposes, but this would be difficult. The ratios of pheromone component precursors produced in yeast in this study are much different from what is seen in the D. saccharalis females, differences that are expected considering the two very different biological systems and their possibly incomparable gene expression levels. It would be most efficient to develop biotechnological production of the major and also most complex structure of the blend components, for possible use in mating disruption. The minor components might be produced more easily by already published biological production platforms, especially 16:acid and Z9-16:acid as they are native products in relevant heterologous platforms (Holkenbrink et al. 2020; Xia et al. 2020). A platform for production of Z9,E11-16:OH alone would facilitate down-stream processing, where the diene could be purified from saturated—and monounsaturated products by urea-complexation (Hayes et al. 1998). It would be difficult to isolate Z9,E11-16:acid from its isomers, and there are many examples of geometric isomers or pheromone analogues that disrupt pheromone communication by acting as behavioural antagonists (Eizaguirre et al. 2007; Juárez et al. 2016; Wang et al. 2022; Witzgall et al. 1999). Purification from the isomers might not be necessary for D. saccharalis IPM, as they don't elicit any antennal responses in GC-EAD experiments (Batista-Pereira et al. 2002).

Despite the observed deviations of the yeast extract from an optimal D. saccharalis pheromone blend, it might still be active for IPM purposes, as seen for a crude extracts of the codling moth (Cydia pomonella) pheromone produced by engineered oilseed plants Camelina sativa (Xia et al. 2021). Here traps with crude pheromone plant extract still caught males, although not at levels of purified extracts or synthetic pheromone. Field tests with sticky traps baited with live D. saccharalis females were already in the 1960s shown to reduce the damage on sugarcane in Louisiana (Hammond and Hensley 1971). However, it has also been shown in the field that traps baited with Z9,E11-16:Ald alone is much less attractive to males compared to live females (Svatos̆ et al. 2001), and the newly identified four-component blend (da Silva et al. 2021) has shown similar inferior attractiveness (P. H. G. Zarbin, unpublished data). Research on optimisation of component ratio and dosage in trap lures could improve attractiveness in the field. The low trap attractiveness could also be explained by a pheromone blend that is still incomplete, or enhancing or synergistic effects of compounds present in the gland extract (Hall et al. 2017; Meier et al. 2016) that don't necessarily elicit detectable antennal responses in GC-EAD analyses for this species. Pheromone blend components could remain elusive because this species show low electrophysiological responses to minor pheromone components, as observed by Batista-Pereira et al. (2002) to Z11-16:Ald, and also by Kalinova et al. (2005), where >100 ng was needed for a significant response as opposed to <1 ng of Z9,E11-16:Ald. Biosynthesis studies such as the present could aid in elucidation of putative missing blend components, in the form of specific precursors observed in the pheromone gland and/or genes showing female-biased expression and a specialised function or producing minor by-products related to the major pheromone components. Further studies will have to be carried out to reveal any unknown components in the pheromone blend, compounds that are possibly produced in very low quantities in the pheromone gland but may be crucial in eliciting male attraction to a synthetic blend comparable to that of conspecific females.

Mating disruption has not been assessed for this species, and might still work relying on only the major pheromone component, as seen for other lepidopteran pests, for example the European grapevine moth (Lobesia botrana) (Ioriatti et al. 2011). Mating disruption based on biotechnologically produced sex pheromones, in a green and cheap production, can be the way forward in controlling D. saccharalis in the vast Brazilian sugarcane fields. Half of the cultivated land area in Brazil is covered by sugarcane crops (FAOSTAT 2021; Parra 2014), and it's a crop important for both sugar- and ethanol industries, smallholder farmers and as a renewable biomass (da Cunha Borges Filho et al. 2019; Dias and Sentelhas 2018; Goebel and Sallam 2011). Because D. saccharalis damage has a significant economic impact and is difficult to control using conventional insecticides, it's pertinent and pressing to develop IPM technologies to control this pest.

The above description is given by way of example, and not limitation. Given the above disclosure, one skilled in the art could devise variations that are within the scope and spirit of the invention disclosed herein, including various variations of Dsac_NPAQ. Further, the various features of the embodiments disclosed herein can be used alone, or in varying combinations with each other and are not intended to be limited to the specific combination described herein. Thus, the scope of the claims is not to be limited by the illustrated embodiments.

SEQUENCE LISTING:

| | |
|---|---|
| SEQ ID | ATGGCTCCGAATTTGATAAAAAACGAAGGAACCTT |
| NO: 1 | TGATGTGGATATAAAACCAGACGAACTGCAGCTGC |
| | CTAAATTTTCCAATATTAAACCTAAAATATTGCAT |
| | GTTAATTTGATATATTTCCTTTACTGGCATTTGGC |
| | AGCGCCATATGGATTATATCTATGTTGTACATCAG |
| | CTAAATGGGCAACAATAATATTTGGTTTCGTCATG |
| | TTCCTGGTAGCAGAACTGGGCATAACGGCCGGTGC |
| | ACACAGACTCTGGTCCCACAAGAGTTACAAGGCGA |
| | AATTGCCACTACAAATAATTCTAATGTTATTCAAC |
| | TCGATAGCGTTCCAAAATTCCATAATACATTGGGC |
| | CAGAGACCACAGACTTCACCATAAATATTCTGATA |
| | CCGAGGCAGACCCTTATAATTCGTCTAGGGGATTT |
| | TTTTATTCGCACTTTGGATGGTTGATTACTGAGAA |
| | AAATAAAGAGGTTTTAAATAGAGCGAAGAGCATAG |
| | ATGCATCGGATTTGTATAACAACCCGGTACTGCAG |
| | TTCCAGAAAAAATACGCAGTACCAGTTTTTGCATC |
| | GTTATGTTTCATATTGCCGACGTTAATACCCATGT |
| | ATCTATGGGGAGAATCTTTCAACAATTCCTGGCAT |
| | GTCAACCTGCTGAGATACATTATGAATTTGAATGT |
| | TACACTTCTTGTTAACAGCGCTGCCCATAGATGGG |
| | GTTATAAGCCATACGAAAAAAACATTAATCCAACA |
| | CAAAATGTCTACGTTTCTTTTGTCACACTCGGCGA |
| | AGGATTCCATAATTACCACCATATTTTTCCATGGG |
| | ATTACAAAACGGCAGAGCTCGGTAACAACAAGTTA |
| | AATGTTACCACTTGGTTTATAAATTTCTTTGCGAA |
| | TATTGGCTGGGCTTATGATCTCAAAACTGTGTCAA |
| | ATGAAATGGCGAGAGCAAGAGCGATGAGAACTGGT |
| | GATGGAAAAGATTTATGGGGCTGGGATGAGAAAGA |
| | TGTAACTGATAAGGATAAAGAACACACAGATATTT |
| | TAAACGGTAAAACGGATTGA |
| SEQ ID | ACTACAAATAATCCTAATGTTATTCAACTCGATAG |
| NO: 2 | CGTTCCAAAATTCCATAATACATTGGGCCAGAGAC |
| | CACAGACTTCACCATAAATATTCTGATACCGAGGC |
| | AGACCCTTATAATTCGTCTAGGGGATTTTTTTATT |
| | CGCACTTTGGATGGTTGATTACTGAGAAAAATAAA |
| | GAGGTTTTAAATAGAGCGAAGAGCATAGATGCATC |
| | GGATTTGTATAACAACCCGGTACTGCAGTTCCAGA |
| | AAAAATACGCAGTACCAGTTTTTGCATCGTTATGT |
| | TTCATATTGCCGACGTTAATACCCATGTATCTATG |
| | GGGAGAATCTTTCAAGAATTCCTGGTATGTCAACC |
| | TGCTGAGATACGTTATAAATTTGAACGTTACATTT |
| | CTTGTTAACAGCGCTGCCCATAAATGGGGTTATAA |
| | GCCATACGAAAAAAACATTAATCCAGCACAAAATG |
| | TCTACGTTTCTTTTGTCACACTCGGCGAAGGATTC |
| | CATAATTACCACCATATTTTTCCATGGGATTACAA |
| | AACGGCAGAGCTCGGTAACAACAAGTTAAATGTTA |
| | CCACTTGGTTTATAAATTTCTTTGCGAATATTGGC |
| | TGGGCTTATGATCTCAAAACTGTGTCAAATGAAAT |
| | GGCGAGAGCAAGAGCGATGAGAACTGGTGATGGAA |
| | AAGATTTATGGGGCTGGGATGAGAAAGATGTAACT |
| | GATAAGGATAAAGAACACACAGATATTTTAAACGG |
| | TAAAACGGATTGA |
| SEQ ID | ATGGCTCCGAATTTGATAAAAAACGAAGGAACCTT |
| NO: 3 | TGATGTGGATATAAAACCAGACGAACTGCAGCTGC |
| | CTAAATTTTCCAATATTAAACCTAAAATATTGCAT |
| | GTTAATTTGATATATTTCCTTTACTGGCATTTGGC |
| | AGCGCCATATGGATTATATCTATGTTGTACATCAG |
| | CTAAATGGGCAACAATAATATTTGGTTTCGTCATG |
| | TTCCTGGTAGCAGAACTGGGCATAACGGCCGGTGC |
| | ACACAGACTCTGGTCCCACAAGAGTTACAAGGCGA |
| | AATTGCCACTACAAATAATTCTAATGTTATTCAAC |
| | TCGATAGCGTTCCAAAACTCCATAATACATTGGGC |
| | CAGAGACCACAGACTTCACCACAAATATTCTGATA |
| | CCGATGCTGACCCTCATAATGCGTCTAGAGGTTTT |
| | TTTTATTCGCACGTCGGATGGTTGATTACTAAGAA |
| | AAATAAGGAGGTTTTAAAAAGAGGAAAGAACATAG |
| | ATGCATCGGATTTGTATAACAATCCAGTACTGCAG |
| | TTCCAGAGAAAATACGCAGTACCAGTTTTTGCATC |
| | CTTCTGTTTCATATTGCCTACGTTAATACCCATGT |
| | ATCTATGGGGAGAATCTTTCAAGAATTCCTGGTAT |
| | GTCAACCTGCTGAGATACGTTATAAATTTGAACGT |
| | TACATTTCTTGTTAACAGCGCTGCC |
| SEQ ID | ATGGCTCCTAATGCAACAGACGTCAATGGCGTATT |
| NO: 4 | ATTCGAAGATGATGCCGCTACTCCGGATATGGCGT |
| | TGCCAAATCTACCTGTACAAAAAGCCGATAACTAT |
| | CCCAAAAAATTTGTATGGAGAAACATCATAGCATT |
| | CGCATACTTGCATCTCGCCGCAATCTATGGAGGAT |
| | ATCTATTCTTGTTTTCTGCAAAATGGCAAACTGAT |
| | ATATTTGCTTACATTTTATATGTCATGTCAGGACT |
| | AGGAATTACTGCTGGAGCCCACAGACTTTGGGCAC |
| | ATAAGTCTTATAAGGCTAAATGGCCACTGAGACTT |
| | ATTCTAGTTTTCTTCAACACTCTGGCTTTTCAGGA |
| | CTCTGCAATAGATTGGTCACGTGACCATAGAATGC |
| | ATCACAAGTATTCAGAAACCGATGCTGATCCACAC |
| | AATGCAACCCGCGGGTTTTTCTTCTCCCATATTGG |
| | CTGGCTGCTTGTTAGGAAGCATCCTGAACTCAAGA |
| | AAAAGGGCAAGGGGCTTGATCTAAGTGATCTCTAT |
| | GCGGACCCAATACTTCGTTTCCAGAAAAAGTACTA |
| | CCTAATTTTAATGCCCCTCTGTTGCTTTGTCATGC |
| | CAACAGTGATACCAGTATATTTCTGGGGTGAGACT |
| | TGGACGAATGCATTCTTTGTAGCTGCCCTGTTCCG |
| | TTACACATTCATTCTTAATGTCACATGGCTTGTCA |
| | ACTCTGCTGCCCACAAATGGGGTCACAAACCCTAT |
| | GATAAGAACATTAAGCCTTCTGAAAATCTCTCTGT |
| | TGCAATTTTTGCTCTTGGTGAAGGCTTCCACAACT |
| | ACCACCATACATTCCCCTGGGATTACAAAACGGCT |
| | GAACTAGGAAATAATAGACTAAATTTCACAACTAA |
| | CTTTATTAACTTTTTCGCAAAAATCGGCTGGGCTT |
| | ATGATATGAAAACAGTTTCTGATGAAATTGTACAG |
| | AAGAGGGTTAATCGTACAGGAGATGGTAGCCATCA |
| | CCTTTGGGGATGGGGTGATAAAGACCAGCCCAAAG |
| | AAGAAGTGGATGCAGCTATCAGGATCAATCCAAAA |
| | GATGATTAA |
| SEQ ID | ATGGCTCCAGACTCAGAAAAAAGACAAATCAGTTT |
| NO: 5 | TCCGCAGCTCTACTACCCAGCGGATAGGGACACAC |
| | TTCCCAAATTACCTTCGCAATGGTTGAGTGGTAAA |
| | AAAGTACATGATGGCGCCGAAGACCTGTGGAGGAT |
| | ATACGATGGCCTCTACGATTTGACTACATTTATAT |
| | CAACACATCCTGGAGGTCCTTTTTGGCTGACTTGT |
| | ACGAAAGGAACAGA |
| | TGTAACAGAAGTATTTGAAACACATCATCTTAAAG |
| | GAATCGCCGAACAACTGCTGCCTAAATATTTTGTA |
| | AGAAAAGCGGCGACACCTAGAAATTCTCCATTTAC |
| | TTTTGAAAGCGATGGTTTCTATAAAACCCTGAAAT |
| | CCAAAGTTATGGAGAATTTACATATCATACCCAAG |
| | GAGGAAAGGAAAAAGAGTGACACTATAGTCACCTA |
| | TTTACTAATGGCATTTATTATTTTAAGTCCACTAA |
| | GTTGTTGGGTGACGCCTGACAATTTACTTTTAGGA |
| | GCAACATTAATATTTTTAAATGGTTATGTCTTATC |
| | TGCATTAGTTTGCTGCGGCCATAACTATTCTCATA |
| | GGGCGGATAGTTGGCGAATGTATTTGATGAACCTC |
| | AGTGGGATGGGTAATGATGAATGGCGGATATCGCA |
| | TGTTCTGTCACATCACACACATACCAATACTGTAA |
| | ACGACGTGGAACTGAGTATACTAGAACCATTTCTT |
| | CAATATCTTCCGTTAAGAGACAAGCCTATTTGGGC |
| | ACAAATGGGAGCATTCTACTGGCCTGTTATATACT |
| | CATTTGCCTTTTTAAATGGACTTATTAAAGAGACT |
| | ACGGCAGCGATTACAAAATTTGAAGGCAAATCTTT |
| | GACGTGGGTGTATATTATACCTTTTTTTCTTCCTA |
| | CATGGATGTGGTTAGCTGGTGGGTTGCCGTTACTT |
| | TGGACTCTGGCTATCTGGCAAGTCACGAATGTGGT |
| | GGCGAGTTTCTTTTTCGTATTTTTCGGCTTGACCG |
| | CTGGTCATCATGCACATACAAATTTCTTCGAGGGA |
| | GACACACCAAGGAAGGAATATTTGGATTGGGGCAT |
| | ACATCAATTGGATACAATAGTTGAGAGAATAGATT |
| | ACGCAACGGATCATTTTAAAGCACTCACACGTTTT |
| | GGTGATCACGGTCTTCACCACATGTTCCCGACATT |
| | GGACCACGCAGAGCTAAAATACTTATATCCAATTT |
| | TTATAGACCACTGTAAGAAATATGAGACTGAACTT |
| | CGTATGACCACGTTTTATCAATCAGTCATCAGTAA |
| | CGTTAAGCAAATAATTAGAAAAAGACCCCAAGATT |
| | TGAGGAATACATCGCGGCGATCAAACGGCATTGAA |
| | AATAATTTCTTTATGTATAAAAAATAG |
| SEQ ID | ATGTCCGAAGAAACGAAAAATAATCAGCTTAGCAA |
| NO: 6 | AGCGAGGGAAGCCGACTGGCCAGCTGTACTTTTCT |
| | TCATTCACGTCCATCTGTTGTCGGGTTACGGCCTT |
| | TGGCTTTTATTCTATGAAGTAAAATGGATGACTTT |
| | AATATTTCTTATAGTATTGACTTTGGTAGCGATGT |
| | TGGGAGTCACTGCGGGGGCGCATCGTTTGTGGGCA |
| | CACCATTCTTATCAAGCCAGCACAGGACTACGAAT |
| | CATTCTAATGATCTTCCAGACACTAGCCGGTCAGG |
| | GTTCGATATACAATTGGGTTCGACACCATCGGCTT |
| | CATCATGCACATTTCGGAACAGACGGTGATCCATA |
| | TGACTATAAAAGAGGGTTCGTACAATCGCATGTGG |
| | TCTCACGCCTCCGTCGACTCAGTCCTTATCAGTCT |
| | CAACTGATTAGTGAAATCGACATGTCTGACTTGGA |
| | GAATGATCCTGTTATCATGGTGCAAGATAAATTGT |
| | ATTGGGTGTTATACTCCATTATATTTCTTCTTCTA |
| | CCACTAAATGCACCGCTTGAATACTGGAGCGACAC |
| | AGTGCTCAGTTCGGTATTCGTTATTGGCTTTCTTC |
| | GTTACGGACTGGTGTTGCACGCATCGTGGCTTGTT |
| | GAAAGTAGTATCTGCGTTTGGGGTTTGAAGCCTGG |
| | TGAGAAATGCCCACCTGACTCTAACTGGATTTTCG |
| | TGCTGTCAAAGTCGTTATGGCCCCATTACCATTAT |
| | TTGATCCCATACGACTATAAGTCTGGAGAGTACGG |
| | AACATACGATTCAGGATGCACATCAGCATTCATTC |
| | GAGTGTTCGCTGCATTGGGCCTAGCTACAAATCTG |
| | AAAACAGTAGATACTGCCACAGTACAGAAGGCTCT |
| | TGCTCACTCCGCCCGATCGAAACTCCC |
| | AGTTCAGACGTGCTTGGAGGAAGCTATATCAAAAC |
| | AAATTCTACACGAAGAACATTACTTGCGTAGAGGG |
| | TGA |
| SEQ ID | ATGGGTGCCAAAGTGTCTAGGACTGATTTCGAGTG |
| NO: 7 | GGTTTACACCGAGGAACCACACGCTAGCCGCCGGA |
| | AAATCATCTTGGAAAAATACCCGGAAATAAAGCAG |
| | CTCTTTGGCTACGACCCTCTATTCAAATGGGTGGT |
| | GACTGCTATGGTGATCACTCAGTTGATCACCTTGC |
| | CTATAGTGCAACACCTCTCCTGGCCGATGACCATG |
| | CTGATGGCTTACTGCTTCGGTGGAGTGATCAACCA |
| | TTCCCTCATGTTAGCCATACACGAAATAGCTCACA |
| | ACCTGGCGTTTGGTCACAATCGACCATTTCACAAC |
| | CGGCTCTTTGGTTTCTTCGCGAATATTCCAATCGG |
| | CATACCGATTTCAATAAGCTTTAAGAAATACCACC |
| | TGGAGCACCATAGGTATCAAGGTGATGAAGTGATT |
| | GATACAGATCTACCTACACTTATAGAAGCTAAATT |
| | GTTTTGCACCACCGGTGGCAAGCTGCTGTGGCTGT |
| | TCCTCCAGCCTTTCTTTTACGCTGTCAGACCTCTG |
| | GTGGTTAGACCTAAACCGCCGACACCTCTGGAACT |
| | GATCAACCTGGTGATACAGTTGTTCTTCGACGCCA |
| | TAGTTGTGAAATTGTTCGGTTGGAAGGTGTTGGCT |
| | TATTTAGTACTTGGATCGTTAATGGCGATGGGTGT |
| | ACACCCTGTAGCTGGTCATTTTGTCTCCGAACATT |
| | ATATGTTCAAGAAGGGCTTCGAGACATACTCGTAT |
| | TATGGACCCCTGAATTGGATAACTTTCAACGTTGG |
| | CTACCATAATGAACACCATGACTTCCCAGCGGTGC |
| | CCGGACGGAGATTACCAGAGGTGAAACGCATAGCC |
| | GCTGAATTCTACGACAATCTGCCGCAACACAACAG |
| | TTGGTCCAGTGTCCTATACGATTTCGTGACTGACC |
| | CCGACATTGGCCCGTACGCTAGGATCAAACGCAAA |
| | CATGTAGGCTTAGATAGCTAG |
| SEQ ID | ATGGCTCCAGCGCAACAGGACGTTATGCTGTGTAA |
| NO: 8 | AGAATCGGTGCAGTCAAAAATAAAAATAAGACATA |
| | TAGATAGTGAATATAAACGTGAAAACGGTGATGTC |
| | AGTGAAAATAACAACGTTGTGGTGAAAAATGGTGC |
| | CAGTGATGAAACAACCAGTGATAAAGACTTCGATT |
| | TGAGCAAATATGAAGCGATGAAGTTCACTGCACGA |
| | ATAAAGTGGCCAGACCTTATAGTTCAATTGTCATT |
| | ACATTTAGTTTCATTATATGGACTTTATCTAATTT |
| | TAACATTTGAAGTGAAAATCCTCACAATTTTATTT |
| | GTGTTAGCAACAATATACACGTCGGGGTTCGGCAT |
| | TACGGCCGGTGTACACAGACTATGGTCGCATCGAG |
| | CGTACCGAGCTAAGCTGCCGCTGCGGATACTCCTC |
| | GCCTTCCTATTCACAATAACTGGACAGCGCGACAT |
| | ATATACCTGGGCTCTAGATCACCGGGTACACCACA |
| | AGTATTCGGAGACGGTGGCTGATCCTCACGACGTC |
| | AGACGCGGCTTCTGGTTCGCGCACGTCGGTTGGCT |
| | AGTACTGACTCCCCATCCCGCTGTGGAGAACCGCC |
| | GCGCCGCATTAAAGCTCACTGCTACAGATCTCATC |
| | GCCGATCCTGTCGTCAGGATACAGAAGATAACTTT |
| | CATACCACTTTTCGCACTTCTGAACATAGTGGTGC |
| | CTACAGCGGTGCCGTGGTACTGCTGGCAAGAGACC |
| | CTGTTCAATAGCTTTGTGCTCAGTTTTGTCACTCG |
| | ATTCACCATCACCTTAAATATAGCTTACTGTGTCA |
| | ACAGCTTCGCACATCTGTGGGGCAACAAGCCTTAT |
| | GATAGATTTATCAAATCTGTGGAGAACAAAGCGGT |
| | GAGTCTGGCAGCCTTAGGTGAG |
| | GGTTGGCACAACTACCATCACGTGTTCCCCTGGGA |
| | CTATCGAACCTCTGAACTGGGGAGGATCAACATCT |
| | CCACCAACTTCATCGATGCCTTCGCAAAAATTGGC |
| | TGGGCATATGATCTCAAAGCTGCAAGTAACACAAT |
| | GATTATCAACAGAGCTAAGAAATCAGGAGATGGAA |
| | CGTTTGGAGAAACCGAAGAGCCAGATCCGAATCCT |
| | TCAGAATATAAACACTTGTAG |
| SEQ ID | ATGGCACCAAAAAATTTGGAATATTTAGAAATCGC |
| NO: 9 | TCAGAAGAGAGCCTTTGAGAAGAAAACTCATGTTA |
| | GTTTTCCTCAACTTAAATATCCCTCCCTCAGAGAT |
| | ACAGGATTTAGAGATCCAGTGCAATGGCTGACTGG |
| | AAAAGCTTTGGACGATGGAGCAGAAGGCTTGTGGA |
| | GAATTCATAATTCTATTTATGATCTTACTAGTTTC |
| | ATCGATAACCATCCCGGTGGTGCAGAATGGCTAGA |
| | ACTCACAAAGGGAACTGATATAACAGAGGCATTTG |
| | AAGCTCATCATCTTAACCCTGTAGTAGAAAAGATG |
| | TTAGATAAATATTTTGTAAAAAATGCAACAACGCC |
| | AAGAAACTCCCCTTACACGTTCAATGAAGATGGTT |
| | TCTACCGTACACTTAAAAATTCTGTGAGAGAAGAA |
| | TTGAAAAAAGTACCAAAGAACATTCCTAACCATGC |
| | AGACAGAATCATTGATGGATTATTTATTACATGTT |
| | TGGCAACAGCCGCATTGACTTGTTGGGCGAACAAT |
| | TATTGGGTTGTGATGTTATCATATCTAATCTCTTC |
| | TTTAACATTAGCATGGACTGTTGTTGCATCACATA |
| | ACTATATACACAGGAAAACCAATTGGAGAATGTAC |
| | ATTTTTAGTATGTGCTTATGGTCATACAGAGATTT |
| | CAGGGTGTCCCATGCTCTCTCACATCACTTATATC |
| | CAAACACTCTAATGGATTTAGAGATAAGTGCATTT |
| | GAGCCAATTATACAATGGAATCCGCGGCCAGACAA |
| | GCCGTTTTTCACATACTTTACTTTTTTGTTTCAAT |
| | GTTTATTATATCCCTTTGCCTTCATGATGAGTTTT |
| | GTGAAAAGATTTCTTCTCAACTTTGTAAGAAAAGA |
| | TTTTTTCAAAAACCATTATCGCTGGCATGATGTAA |
| | TCGGTCTTCTTCTTCCCGTTTGGATGTGGGTCGTC |
| | AGCGGATGTTCCTTCAGTGACGCTATTATCAACTG |
| | GTTATGGATCAATTGCACTAGCAGTTTTATCTTCA |
| | TGATGATCGGTACCAATGCAGCACATCACCACCCA |
| | CGAATATTCAAAGATGGTGACGAAGTCAGGACAAT |
| | TACACCTGACTGGGGAATGCACCAACTTGAGGCCG |
| | TGATGGATCGTAATGATATCAACGGGAGCCTTTTC |
| | AAAGTTATGACCTTCTTCGGTGATCACGCTCTTCA |
| | TCATCTTTTCCCAACTCTAGATCATGCCGTGTTAC |
| | CATATTTGTATCCTGTATTTTTGGAACACTGTGAA |
| | AAATTCAAAGCAAACTTCAGACTTACGTCACAATT |
| | TGATCTTTTTATTGGTCAAATCAAGAGGGCAAATG |
| | AAAAGCAATTCAAACTGTTAGATGATTAA |
| SEQ ID | ATGACCTCTTCTTTGCTTCTGGCGAGCACGATCTT |
| NO: 10 | CACAAAAGATATCAAACAAGTAGAAGATGACATGC |
| | CGCAAATTTCTACACGTCTGTCGAGCGGGAAGAAC |
| | AGAGATTATGAATGGCAGATAGTATGGAGAAACGT |
| | ATTAGGGTTCGTCTATCTCCACGCGGCAGCATTTT |
| | ATGCAATTTATTTAACAATAACAGGCAGATTCAAA |
| | TTATATACTTACATATTTGCTTTAACTTTTGCTAT |
| | ACTCGCTGCTATGGGAGTAACGGCGGGAGCACATA |
| | GGCTATGGGCTCATCAGGCGTACAAAGCAAAATGG |
| | CCACTAAGACTGTTCCTAGCCGTTTTGCAAACTAT |
| | GGCCTTCCAGAATCACATTTACGAATGGGTGCGAG |
| | ATCATAG |
| | AGTTCATCATAAATTCACGGAAACTGACGCTGATC |
| | CCCACAACGCCAAGCGTGGTTTCTTCTTCTCCCAT |
| | ATCGGCTGGCTGATGATTCGCAAACATAAAGACGT |
| | GTTCGAGAAGGGTATCACCATTGACATGTCCGACT |
| | TGGAGAAAGATCCTATCGTCATGTTTCAGAAAAGA |
| | ACATATTTGTTCGTGATGCCACTTCTCTGCTTCAT |
| | AATACCCGCATGGATCCCCATTTACTTCTGGAACG |
| | AGGATCCTTGGACATCGTGGTATATTGCAGCTATA |
| | TGGCGTTATACACTATCCCTTCACTTTACTTGGCT |
| | CGTCAATTCCGCTGCTCACATATGGGGAAATAAAC |
| | CATACGATAAATATATAGGCGCAACTGACAATAAA |
| | ACTGTTGCTATTTGCGCATTTGGTGAGGGTTGGCA |
| | TAACTACCACCACGTGTTCCCATGGGACTACAAAG |
| | CGGCAGAACTGGGAAATTATTCAACAAACATGTCA |
| | ACGGCGTTAATTGATCTTGCTGCAAATTGGGGTTT |
| | GGCTTATGATCTGAAGACGGTGTCTGTAGAAATGA |
| | TAAAGAAACGTGCAGCAAGAACAGGGGACGGCACT |
| | CATCCATCTAATAAAGACACCAATGCATCTCACGA |
| | AGATCATCACCATCCAGAAAATCCTGTCTGGGGCT |
| | GGGAAGATAAAGACATGCCAGAAGAAGATAGAAGA |
| | TACGCAGAAATTCATAAAAAAGTAGAATGA |
| SEQ ID | ATGGCGCCTAAAGATCCCGAATGGGAAAGTGAAGC |
| NO: 11 | TCTAAAAAGGATAGATGAGAAAAGAACTCATGTCA |
| | GTTTCCCAAAGTTAAAATATCCGTCTTTAAGGGAT |
| | GATGCTATGAGGGATCCTATACAATGGCTTACAGG |
| | AAAAGCTATGGATGATGGTGCAGAAGGTATATGGC |
| | GTATTCATGATAAGTTGTATGATTTTACAAGTTTC |
| | ATGAAGCGACATCCCGGCGGCGAAGAATGGTTGGA |
| | GTTGACGCAGGGCACTGACATAACAGAAGCGTTCG |
| | AGGCCCATCACATAAATCCGACAACACAAAAACTC |
| | TTAGACAAATTCTACGTCAGAGACGCAAACACACC |
| | AAGGAACTCCCCTTTCACGTTTAAGGAGGATGGTT |
| | TTTATAGAACTCTGAAAAAGGCGGTGTACAAAGAA |
| | TTGGAGAAAATACCCAAAGATGTATCTAAAGCTGC |
| | GGATAGGATAACAGATTGGCTATTTGTATCTCTTT |
| | TATGTAGCTCCGCAATGGCAGTCTGGGTGGAAAAT |
| | ATCTATGCTGCGACCGTTTGGTACATTTTTGCATC |
| | CGTAAATCTAGCATTTTTGACGGTGGCATGTCACA |
| | ACTATATTCATAGGCGTACGAATTGGAGGATGTAT |
| | TTGTTTAATATGAGCATGTGGTCTTACAGAGATTT |
| | CCGTGTGTCCCATGTGATGTCACACCATCTTTACA |
| | CGAACACTTTAATGGACTTGGAGATCAGTGCACTG |
| | GAGCCAGTTTTGTTCTTCAATCCAAGGAAAGATAA |
| | GCCGATGTACGCACGACTGGGTTTCATAACTGAAT |
| | TGTTCTTCTTTCCGTTCGTTTTCCTCATAAATTTT |
| | ATGAAAAGATTCATATCAGTGTTCATCAGAAAAGG |
| | ATTTTTCAAACGACATTATCGATGGCATGATATGA |
| | TAGGATTTCTCCTGCCATTTATTATGTGGGTCGCA |
| | AGTGGTGCGTCAATCCTTCACGTTCTGTATTATTG |
| | GCTTTGGATCAACTGTACAGGAAGTTTAATATTTT |
| | ATTGTATAGGCGTAAATGCGGCACATCATCACCCT |
| | GAAGCTATCAAAGATGGTGATAAACCAAGGAGTGA |
| | AACACCAGATTGGGGTGAACACCAAGTGGAAGCTC |
| | TCTTAGACCGCAAAGATGTGAATAACAACGTCTTC |
| | GCCGTGGTCGTGTTATTCGGGGAGCATGCCCTACA |
| | CCATATGTTTCCGACACTAGATCATGCGGTGTTGA |
| | AATATTTACACCCCATCTTCATCGAACATTGCGAG |
| | AAATTCAAGGCAAATTACAGAGTATCGACG |
| | CAGTTCCTAATGGTTTTGGGACAGATTAAGGAATG |
| | TATGCGTGCAGAATTTAGGATTGTTTGA |
| SEQ ID | ATGCCGCCACAAGGACAAGAGCAAGATTCCTGGGT |
| NO: 12 | GCTAAACGAAACTGATGTCAAAGACCAAGATGTCA |
| | ATCATCTGGTGCCTCCATCAGCTGAGAAAAGGAAA |
| | ATGGATATTGTATGGAGGAATGTGATCATCTTCGC |
| | CTACCTCCACTTAAGCGCCATATATGGTGCATACC |
| | TTTTCTTCACAACTGTCATGTGGAAGACTATGCTC |
| | GCTACTTATATCCTGTACGTCATGTCGGGCCTCGG |
| | GATCACAGCCGGCGCGCACAGACTTTGGGCACACA |
| | AATCCTACAAGGCAAAGCTACCATTGCGTATCTTA |
| | TTAACTTTCTTCAACACAATGGCTTTTCAGGATTC |
| | AGTTTTGGATTGGGCTAGAGACCATAGGATGCACC |
| | ATAAGTATTCTGAAACTGATGCTGATCCCCACAAT |
| | GCGACTAGAGGTTTCTTCTTCTCGCACGTCGGTTG |
| | GCTGCTCGTAAGGAAACATCCTCAGATCAAGGCTA |
| | AAGGAAACACCATCGACTTGAGTGATCTGTGGGCT |
| | GACCCAGTTCTTCGATTCCAGAAAAAGTACTACAT |
| | GTATCTCATGCCATTGGCTTGTTTCGTCATACCAA |
| | CAGTGTTACCTACCCTCTGGGGTGAGAGCCTGTGG |
| | AATGCTTACTTCTGCACTGCTATCTTCCGCTACGT |
| | TTTCGTCCTAAACAGTACCTGGCTCGTCAATTCTG |
| | CAGCTCATTTATGGGGTGAAAAACCTTACGATAGA |
| | CATATAAATCCTGTAGAAATTAAGACTGTTTCTAT |
| | AGCAGCTTTAGGAGAAGGATTTCATAATTACCATC |
| | ACACATTCCCATGGGATTATAAAACTGCAGAACTG |
| | GGGAACTACTCTTTCAATTACACTAAGCTTTTTAT |
| | TGATACGATGGCTAAAATTGGCTGGGCTTATGATC |
| | TGAAAACTGTTTCACATGAAGTGATAGAGAAACGG |
| | GTAAAAAGAACCGGTGATGGAAGTCATAGTGTGTG |
| | GGGTTGGGGTGATAAAAATATACCCGAAGAAGATA |
| | AAAATGATACTACGCTCATAAATCCAGCCAAGTCT |
| | GAATAA |
| SEQ ID | ATGACACCTCAAGTAGACCCGATACCCAGTGGGGT |
| NO: 13 | TTTGTTCGAGACAGAGACACAAACGGCGGACTTAG |
| | GACTAGACGCCGATGTCTCAAAACTGAAGAACGCT |
| | GCCCCAAAGAAATATGAATACGTCTATTTCAACAT |
| | AGTTTGGTTCATATTTCTCCACACCGTTTCGCTGT |
| | ACGGATTATACCTGGCATTCACATCGGCTAAATGG |
| | CAAACGAACCTCTTTGCATCCGTGATGCACTTAGC |
| | GTGTGCTATCGGAGTAGGCGCTGGATCTCATCGGC |
| | TCTGGACTCACAGAGGATACAAAGCTCGCACGCCT |
| | CTTAGGATTTTACTTATGATCTGGCAGACAATGGC |
| | ATTTCAGGACTGTATATTTGAATGGGCACGCGACC |
| | ACCGCACCCACCACAAGTACGCTGACACAGATGCT |
| | GATCCTCATAACTCGGTGAGGGGTCTGTTTTTCTC |
| | TCACTGCGGCTGGTTATGTTGCAAGAAAAGCCCTG |
| | AGGTTATCGAAGGTGGGAAGAGGATTGACGTAACC |
| | GATCTATACGAAGATCCTGTCGTCATGTTTCAGAA |
| | GAAACACTACATGAAGATGATGCCAATCTTGTGCT |
| | TCGTGTTGCCTACCGTCATTCCCGTGTATTTCTGG |
| | GGTGAAACCTGGCTGAACGCATTCTGCATACCAAC |
| | AATCCTACGTTACACGTTCGGTATCAACGTTGTGT |
| | GGTCTATTAATAGTTTCGCCCACCACTACGGCTTC |
| | CGTCCTTATGACAAATCTCTAAACCCACGTGACAA |
| | TGTCGCCATCTGGATGTTCTGCTTAGAGGGTTTCC |
| | ACAACTACCACCACACCTTTCCTTGGGACTACAGA |
| | GCGACGGAATACCCATTTTACAACCTGCTCACACC |
| | CACGGTCGTCTTCATAGATTTAATG |
| | GCTAAGATAGGTCAAGCATATGATTTGAAGAGTGT |
| | AACGCCTGATATTATAAAACAACGAGCTCTTCGCA |
| | CAGGAGACGGCACCCACAACCTCTGGGGCTGGGAT |
| | GATCCCGAGTTTACTGAAAAATTGAAAGCACAGTA |
| | CAGTGTCATAACTAATGTCGAAAAAAAATTTGATT |
| | AA |
| SEQ ID | ATGCCTCCCCAAGGCCAGTCCCCCTCATGGGTGCT |
| NO: 14 | GGAGGAGTCTGACGCCGCCACCGACGACAAGGATG |
| | TGGCGACCCCGGTCCCCCCCTCGGCTGAGAAGAGG |
| | AAGCTTCAGATAGTGTGGAGGAATGTCACCCTCTT |
| | CGCGTTCCTCCACGTCGGCGCCTTATACGGTGGTT |
| | ACCTGTTCTTTACACGGGCTATGTGGACCACCAGA |
| | ATCTTCACTGTGATTCTGTACGTCATGTCCGGCCT |
| | GGGCATAACGGCGGGTGCGCACAGGCTCTGGGCCC |
| | ACAAGTCGTATAAGGCTCGGCTGCCTTTGAGAATA |
| | CTACTAACATTGTTCAACACGCTCGCGTTTCAGGA |
| | CTCCGTGCTAGACTGGGCTCGGGACCACAGAATGC |
| | ACCACAAGTATTCTGAGACCGACGCCGACCCACAC |
| | AATGCAACACGTGGCTTCTTTTTCTCTCACGTCGG |
| | CTGGCTGCTGGTGCGGAAGCATCCGCAGATCAAGG |
| | CTAAGGGGCACACCATCGACATGAGCGACCTCTGT |
| | GCAGATCCTGTTCTGAGGTTCCAGAAAAAATATTA |
| | CTTGACTTTGATGCCTCTCGTTTGCTTCATCCTGC |
| | CAACCTACATCCCAACGCTGTGGGGGGAGTCACTT |
| | TGGAACGCCTACTTCGTCTCCGCGATCTTCCGCTA |
| | CTGCTACGTCCTGAACGTCACCTGGCTGGTCAACT |
| | CAGCCGCCCACAAGTGGGGAGACCGGCCTTATGAC |
| | AAGAACATCAATCCGGTAGAGACCAAACCAGTCTC |
| | CCTGGTCGTGTTTGGGGAAGGTTTCCACAACTACC |
| | ACCACACGTTCCCGTGGGACTACAAGACAGCTGAA |
| | CTGGGAGACTATTCCTTGAACCTTTCCAAGCTCTT |
| | TATAGATACCATGGCTAAGATTGGATGGGCCTATG |
| | ATCTGAAATCGGTTTCCAAGGACATTGTAGAGAAA |
| | CGAGTAAAGAGGACGGGAGACGGTAGCCACGCCGT |
| | GTGGGGGTGGGACGACAAGGACGTTCCCGTGGAAC |
| | AGAAGACAGCAGCGGTAATAATCAACCCTGAAAAG |
| | ACTGAATGA |
| SEQ ID | ATGGAAGAAATTGAAAACGTAAACGAAATCCTAGT |
| NO: 15 | GAAATCACAATTGAATTCGCCTGTAGTGAACTTCT |
| | ATTCTGGTAGATCAGTATTTATTACAGGCTGTACC |
| | GGATTCTTAGGCACGGTTTTACTTGAAAAACTGCT |
| | GTTTACATGCGGACATAATCTAAAACACGTTTACA |
| | TACTAATAAAACAAAAGGATGACCAGAAAATTGAT |
| | GAAAAGGTGTCAAATTTCTTTAACTCCCGTGTGTT |
| | CCAGAGGTTGAACAAGCACAATCCCAACTTCAGAG |
| | CGAAGATTATACCAATAAGTGGTGATCTTTCGAAA |
| | AAATGTATTGGTATCAATGAAAACAACTTATCTTT |
| | GCTCCGCAAAGAGGTATCCGTAGTGTTTCATTCTG |
| | CCGCAGACATTTCTTTTGATATGAGTGTGAGTGAC |
| | GCTGTCAATATAAACACTAAAGCTACAGAAAAATT |
| | GTTGAAAATTTGCAAAGAGATGCATCAGTTAAAAG |
| | CATTCGTTTACGTATCAACTGCTTACAGTAACTGT |
| | AACAGAAATGTTATAGACGAAAAAGTATACCCTAG |
| | TGATGTACCATTAGAAACACTATACGAAGTACTCG |
| | AGCATTGTAAAAGTGAAAGATTGACTCAATATTTG |
| | CTCAATGGAAGACCTAACGCCTACTCATATTCAAA |
| | GGCACTTTCCGAAGAACTAATACAGAATTACGCGA |
| | ATTCTGTACCATCAATCATCATCCGTCCTTCAATT |
| | ATTATATCATCGGTGAAGGATCCGATGCCCGGTTG |
| | GTTGGGTGGATG |
| | GAATGGCCTCAGTCGAGTGATACACGCGGGGATGA |
| | ACGGAGATATGAAATGTTGGATCGCGGACCCTTTG |
| | TGTACTACAGACATCATTCCTGTGGACTACACAGC |
| | GAATATCATGATAGTTTCAGCATGGGAAGCAGGAG |
| | AAAAATTGAAAAGTAGACATCAGTTAAAAGTATAT |
| | AACTGTTGCTCGGGTCTGCAGAACCCAATTAATTC |
| | TGGAACCATAGTGAAAGATTGTTTGGAATACAACG |
| | AAGAGTATGAGAAAGATAAATCTAAGGTTAACAAG |
| | ATATTTATAATGCGGAATAACTTTTACGTATATTT |
| | AATGTTCCTATTCCTTCATATTATACCTGCTTTCG |
| | TAATTGATGTGTTCTACTTTCTAACAGGAAGAAAA |
| | ATGATAATGTTAAAAAGGCTGAAGAAAATAAAACT |
| | GTTGTCAAAAATGTTCAAATCATTTTGTGTCAAAG |
| | AATTCTTATTCATTGACAATAATGTTCGTAAGCTG |
| | TATGAAAGCTTAAATGAATCCGAGAAGATTTTGTT |
| | CGATTTCAACGTCAAAAATTTACAATGGAAGGAGT |
| | ACATGAAGAGCTTTGTGATTGCGATGAAAGAATAT |
| | AGTAAGGAGACTAAAATCAGCAAAAAACAAAACAA |
| | AATTGAATAA |
| SEQ ID | ATGCCGGATGAATCGCAAGTGCGTGCCTTTTACTC |
| NO: 16 | TGGCAAGAATTTCTTCATAACTGGCGGTACAGGGT |
| | TTGTGGGATTATGTCTAATAGAGAAGATATTGAGG |
| | ACAATCCCAGACGTCGGCAAACTCTACTTGCTAAT |
| | GAGGCCGAAAAAAGGAAAAGACATCGTTGAAAGAC |
| | TGGACGAGTTCCCTAAACACCCGATATTTGAGAAA |
| | CTAATAGAACAAAAGACAATTGACATATTTCACAA |
| | ACTAGTAGCCGTCGCTGGGGATGTTGGTGAAGAGG |
| | ATCTCGGTCTCAGTCCGGAAGACAGGAAAATTCTT |
| | GTTGAAAATGTGAATGTAGTGGTACATTCCGCTGC |
| | CACACTTGATTTCCAAGACAATTTAAGACCAACGG |
| | TACAGATCAATCTACTGGGCACTAGACAAGTTATG |
| | GAACTATGCAAACAAATCAAAAACTTGAAGGTCAT |
| | GATCCATGTGTCTTCAGCTTACGTCAATTCATATT |
| | TAACAGAAGCACACGAAAAAGTATATGAAGCTCCA |
| | GATGATGCGGAAAAAATTATATCTTTAGTTACTAC |
| | ACTCTCTGATGAGGCTTTAGATGAAATAGAACCAA |
| | AGATATTAAAGGATCATCCGAATACATACACATTC |
| | ACCAAACATCTTGCCGAACATGAAGTAAAGAAATG |
| | TTCTGACTTGTTCCCATGTACTATAGTCAGACCTA |
| | CTATGATTGTAGGGTCATGGCAGGAACCAGTGCCT |
| | GGTTGGACATGTTCCAAAGTTGGTCCGCAAGGGTT |
| | CCTTATGGGTGCGAGTAAAGGTGTAGTCAGAAGAT |
| | TACCTCTCTCAAAAGACAACATCGCTGACTATATT |
| | CCGGTGGATGTGGTTGTGAACGAATTACTGGTGGC |
| | TGGATGGCATGCCGCTAAATCTAAGTCTGGTTTGA |
| | CTGTTTATCACTGTTCCTCGTCTACTTGTAGACCG |
| | TTCCGTTGGTTCAGTATAGAGTATTCTCTCAATGA |
| | TAAGTTACATGCATATCCACTGAAGAGTGCAGTAT |
| | GGTATCCATATCTTGCATTTAATTCTTCTTTGTTG |
| | AGTTTTCGTCTGTCGGCCATATTTATACATTTCTT |
| | CCCAGCAATTTTATTGGATTTATTACTTAAGTTAA |
| | CTGGAGGTCGACCCATATTATGGCGATTGAATCGT |
| | AACGTATGGAACTCTTTAGCTCGTCTAGAGAAGTT |
| | CATATTCACCGAGTGGCTATTCCATAATCCAAATA |
| | CTCTAGATCTCTGCAAGCAGTTGAACAAAACGGAC |
| | AGAGAATTATTTTATATTGACATTTCAACTTTGGA |
| | ATGGGAAGAGTACTTTACGAATCTTCAAAAAGGCG |
| | TTAGAAGATATTTAAACAACGAAAAAGAATCTACG |
| | CTCCCAGCTGCTAGGAAAAAGCAAACCAAATTATA |
| | CAT |
| | CTTCCATTTGATCTGGCAAGTGGCCATCATTTCAC |
| | TACTCTGGTACTTCGCTGCCTGCCTGATGGGAGTC |
| | AGTATGATGAAATGCGTGTGGGCAGTGCCCGGTAT |
| | CTACATACTATACTCGTGTTTGTAA |
| SEQ ID | ATGAACCACTCACTGGAGAATTCTCCGCTGATACC |
| NO: 17 | CACCGACATGACCAGAGAGAAGATGGAGAAGTGGG |
| | TGGCAGCGCAACAGAAAGGAGAGAAAATAGACATT |
| | GACATTTATGGTAAACCCACAGAGAAACAAATTAA |
| | AGAGTTGGAAAATATAAGGAATTTGAGCAGAGAAC |
| | TACAGGATAACTTACACGAGTTAGAAAACTCGGTA |
| | CGCATAGCTGACGTGGAAAACCAAGCGATGAATCC |
| | AACGGCACCCATTTTGGATTACTCTGAAGACCACG |
| | AATTCGTGTCTGCCAATAATTTGGACAATTATTAC |
| | GCTGAAGAAGACAAAATTGACGCGAAAGAAGAGGA |
| | GAAGAGACGGTTAACTAAGGGTAAATGTGGCACAT |
| | CAGAGATTCAACAGTTTTATAAGGACCAATCGGTT |
| | TTTTTGACGGGTGGTACCGGGTTTTTGGGTAAAGT |
| | GCTTATTGAAAAACTGCTCCGTACTTGTGGAGACA |
| | TCGACACTATATACGTTTTAATAAGAAATAAGAAA |
| | GGGAAAGATGCCAGAGCAAGATTACATGAAATGTT |
| | GGATGAATTTTTGTTTGAGAAAGCACACGAAATTA |
| | ATCCAAAGGGTATACATAAAGTGGTGCCTATAATT |
| | GGTGATATGGAACTACCAGGCCTTGGAATGTGCGA |
| | AGAGGACAGAAAAACTGTTACCACTAAAGCCACCA |
| | TCATAATAAATGCAGCAGCAACGGTGAAATTCGAT |
| | GAGAAGCTTTCAGTGTCGACTGCTATCAACGTAAA |
| | GGGTACTAAGGAGGTTTTAAAACTAGCTAACGAAT |
| | GCAGAAACTTAAAAGCCATCACACACGTGTCCACT |
| | GCCTTTTCAAATACTCACATTAAATATGTTGAAGA |
| | AAAATTTTATGAGCCCCCTATGTCTGTAGAAGCTC |
| | TGGAAGCTGTGTCAGAAATAAATGATGACATACTT |
| | GACGACATTTTGCCGAGTTTATTAGGAAAACGGCC |
| | GAACACCTACTGCTTCACGAAAGCTGTAGCTGAAG |
| | AAGCTGTCAGGACACACGGGGCAGGTCTACCCATC |
| | TGTATCGTCCGACCTTCCATAATTGTGTCGACATA |
| | CGAGGAGCCAGTACGTGGCTGGACGGACAGCGTGT |
| | ACGGGCCTACAGGACTCGTGATCGGAATCGGCACC |
| | GGGGTGCTCAGGACTATGTATATGGATCTAGACAT |
| | CGTGGCTGACATGGTACCCGTCGATCTAGTGGTGA |
| | ACTCAATATTAGCATCAACGTGGTATACCGCTAAG |
| | AACTACAAAGTGAATCAAACTTCAGATATCCCTAT |
| | TTACAATTTCGTGTCTGGAGCACAGAATCCTATAA |
| | AATGGGGCCAATTTATAGAACTCAATAGGAGATAT |
| | GGCATCGATAAACCCACCACTAAAGCTGTATGGTA |
| | TTATGGTTTGAATCCCACCAATAATTACTACATGT |
| | TCCTGTTCTACAATTTCTTTTTACATTATCTGCCG |
| | GCTCTCCTCATAGATATGTACAGCGCTCTCATTGG |
| | GAGACGAAGAGCTATGTTAAAACTTTACTCGAAGG |
| | TTATGAAGTTGGCCAACATTTTGTTTTACTTCTCA |
| | ACACAAGACTGGAAGTTCTCTGATCGTAATGTTCG |
| | CTCAATGTGGTCGTCACTATCAGAGGCTGACAAAG |
| | CAATATATCCGTTCAGTTTAAGTGAGATGTCATGG |
| | GAGAGGCTTTGCGAGAAGTTTTTAATTGGTTTAAG |
| | AGTGTATCTAATAAAAGACGACCTTTCAACTCTAC |
| | CCGAGGCTAGAAAAAAATGGAACAGGTTGTTCTAC |
| | CTTCACCAAATGCTGAAGACCCTTACAATAGCATT |
| | AATCTTAAACTTAGCGTATCTCGTATTGAGGCCAA |
| | TTTTCACCGCCATACTTGGTTGA |
| SEQ ID | ATGGCGTCCGAGGGTTTCTCAGCGTCTGAGGTGGA |
| NO: 18 | TGGTATGCCGGATCGGATAGCGGAGACGTTTACAG |
| | GCCGTCGCTTGCTGGTCACCGGCGGTACAGGTTTC |
| | ATGGGAAAGGTGCTCATAGAGAAACTGTTGAGAAA |
| | ATGTCCAGACATTAGTCAGATCTTTCTTCTTGTGC |
| | GCACCAAGAAAGGCAAGAATCCGAAACAGAGATTG |
| | GAAGAGATATTCAACGGAGAATTGTTCGAAATGCT |
| | CCGCAACATGAGAGGTGGTATCGAACCACTCTTGG |
| | AGAAGGTGTCGATCATAAGCGGTGATGTGAGTGCA |
| | CCAGACCTGGCCATGAGTGAGGGAGACAGACAGAA |
| | GATCATCGATGAAGTCGATGTTGTAATACACGCTG |
| | CTGCTACCATCAGATTTGATGAAGAGCTGAAGAAG |
| | GCAGTTCTTCTGAACGTACGAGGCACCAAATTGAT |
| | TTTAGAACTCGCAAAACAATGCAAAAACCTACAGC |
| | TCTTCATGCACATATCAACAGCTTACTGTCATCTG |
| | CACGAGAAACTATTGGAAGAAAAGCCGTATCCTCC |
| | ACCCGCTGATCCCCACCAGATTATTCAGGCGATGG |
| | AATGGATGGATGATGAGACCATCGCAACCGTGACA |
| | CCTAAACTCCTAAATAAGCTACCCAACTCGTATGC |
| | CTTCACCAAAGCTTTAGGCGAGGGTCTTGTGGTGG |
| | AATACATGCAGCACATTCCAGCCGTCATACTCCGA |
| | CCTTCCATCGTGATCCCGATATGGCAAGAACCACT |
| | TCCTGGATGGACTGACAATATTAACGGACCTACTG |
| | GTTTGCTGATTGGTGCTGGAAAGGGCGTTATCAGA |
| | ACTATGTACTGCAAGAGCAACAGTTATGCCGACTA |
| | TCTGCCAGTCGATGTGTTCATCAATGGAATCATGA |
| | TCTTGGCTTGGAACTATTTGGTTTGCGGTGATAAA |
| | GAGAGGAACATAGTCAACTTCACGTCATCAGCGGA |
| | GATCAAGGTGACATGGAATGAGCTGATCGAAGCTG |
| | GCAGGGAGATCATCATGAACAGAGTACCTCTTAAT |
| | GGAGTAGTTTGGTACCCTGGTGGTTCTATGAAGCA |
| | TTCTCGTCTGTATCACAACATCTGCGCGTTCTTCT |
| | TTCACTGGCTACCGGCTATATTTATCGATACTCTA |
| | CTCTTTTGTCTTGGCTACAAACCTGTTTTGATGCG |
| | TGTTCACCGGCGCATAAGTAAAGGTTTCGAAGTGT |
| | TCGAGTATTATACAAACAACCAATGGGACTTCAAA |
| | TCTGACACAGCACAGAAAGTGAGAACGAGGATGAA |
| | CGCTCGAGAGAGGCGGGACTATAAAGTTGATGCTG |
| | TTGGCGTGGATATTTCCAAATACTTCGAAGACTGC |
| | ATCAAAGCTGCCAGGATCTACATCCTGAAGGAGTA |
| | CGACGACACTCTCCCGGCAGCCAGGAGACACATGA |
| | GAGTAATGTATTGGGTCGATTTGATTGCACAAATA |
| | CTCTTTTGGGCTCTAATATTGTACTGGATGAAGGG |
| | TCTCTTTTCTGGAATGGGCTCCTTCTTCTTTGGCT |
| | CGAGCAGTGACGTCACGCCATCAGTAATGGCGGCA |
| | TAG |
| SEQ ID | ATGGGCTTTTTAGAAGATAGAGATCTAAGTGATGT |
| NO: 19 | GCCAGGCATCCAGGAATATTTTAAAGGGAAGACTA |
| | TATTCATAACAGGAGGATCAGGTTTTTTAGGAAAG |
| | GCTTTGATAGAGAAATTGCTGTATTCGTGTTCAGA |
| | TTTAGACAGAATCTATATCTTGATGAGGTCAAAGA |
| | AAGGCGTCAAGGCTGAAGATAGACTGGCTGAGCTT |
| | TATTCTGCTATAGCATTCGGTCGTTTGAAAGCGGA |
| | AAAACCGGACATTTTTCAATCAAAAGTTTTTGTTG |
| | TTACCGGAGATGTCATGGAGCCAGGATTGGGTTTA |
| | TCAGAAGAAGATAGAACTCTGTTAGTGAATAGGGT |
| | ACACATAATTTTCCATGTGGCGGCCAGCGTAAGAT |
| | TTGATGATCCGTTGCATTATGCTGCTAAATTAAAC |
| | CTCGGTGGTACCAAAGAGATCGTAGAGT |
| | TTGCCAAAGATGTCCGTAACTTATCTTCTCTGGTG |
| | CACGTGTCGACGTCGTATTCGAACACGAACCGTGA |
| | CGTCATAGAAGAGGTCATGTACCCTCCGCACGCGG |
| | ACTGGAGGGACACGTTACAAGTATGCGAATCTATC |
| | GACGAACAGTCCTTACGCGTGCTGACCCCTAAGTA |
| | TCTCGGTGAATTACCTAACACTTACACGTTTACTA |
| | AACAGCTGGCTGAACACGCGATTTACGAGAACAAG |
| | GGTCTACTCCCAGTCGTCATCATTAGACCATCTAT |
| | AGTGATTTCCAGTGTGGATGAGCCTTTTCCTGGTT |
| | GGATAGAAAATTTCAATGGACCAGTGGGTATACTG |
| | GTTGCTTGTGGAAAAGGTATTATGCGGAGCTTGTA |
| | CACTGATCCTAATTTGATAGCTGACTACATACCTG |
| | TTGATTTCTCTATTAAGAGTATTATAGCTGCTGCA |
| | TGGATTAGAGGTACCAAGGAGTTAGAATCAACAGA |
| | CGATATCCCGATCTACAACTGCTGCGCTGGAAATC |
| | TCAATAACATTACAATGTATGAGCTCGTGGATATC |
| | GGCAAACGATTGGCGGCTAATATGCCTCTTAATGA |
| | CATGTTGTGGAACGTTGGAGGATCCCTTACTACAA |
| | GCAAAACGTTACATTATATTAAGGTGTTGCTGCTA |
| | CATTGCCTTCCTGCCATATTTGTTGATGCACTGTT |
| | ATTGATACTAGGAAAGAAACCAATGTTATTGAAAC |
| | TCCAAAGACGCATTTACATAGCAAACTTAGCGCTC |
| | CATTATTACATAACCAAGCAATGGACGTTCGACAA |
| | TAAAAACTTCGTCCTTCTCCGATCTCGTATAAAGG |
| | AGCAAGACAAAAAGCAATTCTTTTATGACATCGAA |
| | AACGTGGATAAACAAGAATATTTCAGGAAATGTTG |
| | CATTGGAGGAAGGAAATATTTGCTGAAAGAAAAAG |
| | ATGAAGATTTGCCAAAAGCTAAAGCTCATTATGCC |
| | AGAATGTTGATACTGGACAAATGTGTACAGATAAT |
| | ATTTTATGGATATTTAGTCTGGTGGATTTTGAACA |
| | TTGGATATATAAAGAATTTACTAAAATTTGTGTAT |
| | GATATATTTATGCTTTGA |
| SEQ ID | ATGGCGCCACCTGTTAGCGTTAAAGACTATTATGC |
| NO: 20 | GGGGAAATCGATTTTCATCACCGGCTCCACAGGTT |
| | TTATGGGAAAAGTCTTAGTGGATAAGATTTTAAGG |
| | TGCTGTCCGGATGTGAAGAATCTGTATCTCCTCAT |
| | GAGAGCTAAAAAGGGGCACAGCGTTAAGGAGAGAA |
| | TCGACGAGTTTTTGAACTGCAGGGTGTTTGACTAT |
| | TTAAAAAGTGCACAACCAGAGCAGCTGCAGAAGCT |
| | ACAAGTGGTTCCAGGTGACATCTTAGTGGAGAACA |
| | TGGGCATGTCAATGGACGATCGAACAATGTTGCAG |
| | AAGGAATGTCAGATAGTTTTCCATTGTGCTGCATG |
| | CGTTAGATTCGATATGTTTATTCGCGATGCTCTCA |
| | ACTTAAATACCGTTGGAACCCAGAGAGTGTTAGAT |
| | TTAGTCTCCGGCATGACGAAGATAGAGGTCTTCCT |
| | TCACGTGTCGACCGCGTACAGTCGTTGCGAATTAG |
| | ACGTTTTGGAAGAGAAACTGTACCCCTCCAACCAC |
| | AGACCACAGCACGTAATCAACTGCGTCTCCTGGAT |
| | GGACGATGAAATGCTGGAACATTTACAGCCCAAAC |
| | TTATACACCCTCAGCCTAACACGTATGCTTATACA |
| | AAATCATTGACCGAAGATCTCGTGTCACAATATGC |
| | TGGGAAATTTCCAATCATCATAGCAAGGCCATCTA |
| | TTGTAACCTCATCATACAAAGAACCAATGCCTGGA |
| | TGGGTAGACAATTTAAATGGACCTACAGGCCTCAT |
| | GATTGGCGCTGGTAAAGGTGCGATCAGAACTATAT |
| | ATATGGACGAGTCTCTCCGCGCCGATGTTATACCA |
| | GTAGATATAGTAGTCAATGCATGCATACTCCTCGC |
| | TTATTCCACAGGTCTAGAAAAATCGAAAGAGCTGC |
| | AATTTTGTAATTTGACACTGTCAGACGACAACCCT |
| | CTCACT |
| | TGGGGTGCAGCCTTGGATTTTGGTCGTAAACACGT |
| | CACAGAGTTCCCGTCCTCCGTCTGTTTGTGGTATC |
| | CTGGCGGTTCTGCGAAGACATCCTGGTTGCAGCAC |
| | CAAATAGCTGTGTTTTTTACCCATCTCTTGCCGGC |
| | TTATTTCGTTGACCTCCTCTTGATGTTGTTTGGAA |
| | AGAAACCTTTTATGGTGAAATTGCAGAAGAGAATT |
| | AACTACGGCTTACAAGTCATACAATATTATACAAC |
| | GAAGGAGTGGTATTTCAAAAATGACTATTTAAAAG |
| | CTATACGAGAGAAAGTGAGCGCAGAAGACAACAAA |
| | ATATTTTATACTGACACTAAGGTAATCAACTGGGA |
| | TTCATACATCAGGGACTACATTAAAGGCGCACGTG |
| | AATACTGTCTGAAAGAAGACCCAGTGACATTGCCC |
| | CAAGCTAGGAGACTTAATAGGCAGTTATATTATGC |
| | CGATAAATTTTTACAAATGGTCCTGTATTCATTAT |
| | TAACATACGTAATTTATTCATACTTCAAAATGTTC |
| | ATAAGTATGATGAGTGTTTAG |
| SEQ ID | ATGGCATCAGAAACATCAGTGAGGGAGTTCTATAG |
| NO: 21 | AGGCAGGAGCGTCCTGGTGACTGGAGGCACTGGTT |
| | TCATGGGAAAAGTGCTGATTGAGAAATTGCTTTAC |
| | TCCATTCCCGATATCGGCAACATTTATATACTGCT |
| | GCGCCCTAAACGTGGCAAGTCTGTTACGCAGCGTT |
| | TAGAAGACATGCAACGGTTACCATTATTCGACCGT |
| | CTCAAAACCGAACGGCCTAGTGCATTCAAAAAAAT |
| | GAAAGCACTACAGGGTGACGTTCTATTTCATAACT |
| | TCGGCCTCTCTAAAAGCGACATAGAGAACCTGTCT |
| | GCCGAAGTCTCCGTCGTATTCCATTTTGCTGCAAC |
| | GCTGAAACTAGAAGCACCTCTCAAGGATAACGTAG |
| | ATATGAACACCAGCGGTACCCAAAGAACCCTTAAT |
| | ATTGCGAGGCAATTGAAGAACTTAACCATATTCGT |
| | ACATTTGTCAACTGCTTTCTGTTACCCTGACTACG |
| | AAGTATTAAACGAAAAGGTTCACGCGCCACCAGCT |
| | AAGCCGGAAGATGTTATGAGGTTAATTGAATGGTT |
| | AGACGATAAACAAATATCGATACTTACCCCCGCAC |
| | TTCTGGGTCCACACCCTAACTGCTATACTTACTCT |
| | AAGAGGCTCGCTGAAAATATAGTCGAAAACGCTTT |
| | TGAAGAATTGCCTATTGTTATATGCCGACCGAGTA |
| | TAGTATGCCCGTCTTATTCGGAACCGCTGCCAGGA |
| | TGGGTGGACAGCCTGAACGGACCAGTTGGTTTAAT |
| | GTTAGGAGCTGGAAAAGGTGTCATCAGAACCATGA |
| | TGTGTGATGGAAGTCTAGTCGCTCAAGTAATACCT |
| | GTCGATATTGCTATCAACGCAGTTATCGCTGTCTC |
| | CAAAATCGAAGGCAGCAAGAAGAAGAAACCTGAAG |
| | TAATACCAGTGTACAATTTAAACATCGGACATCAA |
| | AAACCAACTACCTGGGGTTCAGTACTTCAAGTAGC |
| | GAAAGACTATGGACGGAAGGCACCACTTGATTGGC |
| | CGTTGTGGTACCCCAACGGAGATATCACCACGAAT |
| | ATGGCATTGCATGAGTTCAGAAGATACTTCTACCA |
| | TCTGATTCCAGCTTACTTGATCGATTTCCTCATGT |
| | TACTATTCGGACAGAAACGATTCATGGTAAGAATT |
| | CAAGACAGAATCAGCCAAGGATTGTACGTGCTCCA |
| | ATACTTCACTACTAGAAACTGGTCTTTCGGCTCCG |
| | AAAACTACGATAGCATACAGAAGGCCTTAAACGAT |
| | GAAGAAAAAGTGATATTCAACACTAACATTGAAGA |
| | TGCTGATCGCGACGAGTACATGCGGCACAGCGTTA |
| | ACGGCGGCCGAGTGTTCTGTTTGAAAGAAGATCCT |
| | AAGAATATAGGCAGGAACAAGATTTATCACAACGT |
| | GCTTTTCGTTCTGGACTGTATCGTGAAGGTCCTGT |
| | TTTGGCTCCTGATACTCTCCTTCGTGGTGTCCTGG |
| | TGCACACCACTGAAGGCAATCTTCTCCATCGGTGG |
| | ACCC |
| | CTGGTCAAACCTTTACCTTTCTTAGGTGCCGCTGT |
| | CTTCGACGGCAATGACTTTTAG |
| SEQ ID | ATGACTTTAGGTGAGAGGCCCCAGATTCCGCAGTT |
| NO: 22 | CTACGCGGGAAGATCGGTCTTCATCACCGGCGCTA |
| | CTGGGTTCATTGGACAAGTGCTGGTGGAGCGACTG |
| | TTGTATACGTGTCCTGATATCGAGAGATTATTCCT |
| | ATTGCTGAGGGAGAAGAAAGACTCTGCGCCTATGC |
| | AACGTTTGCGTTTACTTAAAGATTCGCCAGTGTTC |
| | GACAATATCCGCCAAAAAAACCCCTCGCAGTTGGA |
| | CAAGCTGACCGCGGTGTCTGGAGACATCACGAAGC |
| | CGCAGCTCGGTCTCCAGCGAGAATCAATTGATCTC |
| | CTTCAGAACGTATCGGTAGTACTCCATTCTGCTGC |
| | GACGTTAAAGTTTTCGGAGCCCCTCGCTGCGGCAG |
| | TAGAACAAAACATCAAGCCTGTCATCAAGATTATG |
| | GAGTTGTGTGATAATCTACCCAACATGGAGGCTTT |
| | CGTGTATGTATCAACTGCGTACAGTAACGCCGAGC |
| | TGAGCACGGTGGAAGAACGCGTGTACCCGCCCCCC |
| | GTGCCGCTGAAGGATCTCCTTACTCTCGTAGATAC |
| | CTCAACACCAGAACAACTGGCCGAAGTCACACAAG |
| | AGTACATAGCACCGAAGCCCAATACGTATACATAT |
| | TCCAAGGCTATGGCAGAGGCGGTGGTGCAGGAGCA |
| | CACCAGAAGGACATATTCCGTAGCAATATTCAGGC |
| | CGACTATAGTGGTGTCATCGTTGCGTCACCCGTAT |
| | GCTGGATGGGTGCAAGGACTGAACGGTCCCAGCGG |
| | CGTGGTGGCGAGCGCGGGCAAGGGCCTACTGCACG |
| | TGCAGTATGGACAGCGCACAGCGCGCGCCGACATG |
| | CTGCCAGTAGACATCGCCGTCGACACCCTCATAGC |
| | CGTCGCCTGGGAGACTGCCACCGACAGGACGCCCG |
| | AAGTGAGAGTATACAACTGCAGTACTTGTGAGAAC |
| | CCTACAACTTGGTATCAGTTCGAGGACGGCATAAG |
| | ACGAAATCTGCGCGAGCACCCGTTCGACAACGCCT |
| | TCTGGTGGCCCTGCGGAAGCACCGTCAACAATTGG |
| | TTATTATTCAAAATTCTGGAGTTTCTATTACACAC |
| | GATGCCGTTACATTTAGCTGAGTATGTCATGTCGT |
| | TATTCGGAGCTAAATCAAGGATCAACATGATAACG |
| | TTGAGCGCGCGGTTACAAGGCATGAATTCTGTGCT |
| | GGCTTTCTTTTCCATGCGGGAATGGAAATTCGATA |
| | CACGCAACGTACAAATGTTGAGGAGTAAACTTACG |
| | CCACAGGACGCCGCCATATACAATCTGGATCCTCA |
| | CAGTATAGAGTAA |
| SEQ ID | ATGGACGGAGTCCGTAGCAACGATTGTAAAGAACC |
| NO: 23 | ATTCAACGATAAGGGATTTTGTAACGATACCGCTA |
| | TAATGAATAACGATGTTACATTAGCGAATATAAAT |
| | AACAATAGTTGTAACGACAGATTGAACGGTAATGT |
| | TATTGAAGACGGCAACACTAGTAATATGAGTGAAG |
| | TGCAAAAGTTTTATGATGGGAAAAATATATTAATT |
| | ACTGGAGCTACTGGGTTCCTTGGTAAAATTCTAAT |
| | GGAGAAACTATTGAGGAGTTGTCCGGGTGTAGAAA |
| | ACTTATATTTACTGGTCAGACAGAAGCGTGGCAAA |
| | GACATCTACACGAGAATAGAAGAAATATTTGATGA |
| | TCCCGTGTTCGATCGTCTCAAAGAAGAAGTACCAA |
| | AGTTCAGACACAAGGTGGTCGTCATACCAGCTGAC |
| | TGCGAAGCTGCTGGCCTTGGCCTCACATTATCAGA |
| | CAGACAAATTCTTACTGAGAAGGTGAACATCATCT |
| | TCCACTCCGCAGCCACTGTTAAATTTGACGAGCAT |
| | CTGAGGGCTGCATTTTATACGAACGCACGAGCCCC |
| | ATTACACTTGTTAAGATTAGCGAGGGATATAAAAA |
| | AACTTGACGTTTTGATGCATATATCAACGGTGTAT |
| | TCGAATTCACATTTACAATACGTCGAGGAGAAATA |
| | CTACC |
| | CATGTGATATCAGCTGTGACGAGTTGCAGAAAGAG |
| | ATCGACAAGATGACCGATGCTGAAATAGACAAAAG |
| | TTTACCAAGACTTCTCGGTGCATGGCCTAACACAT |
| | ATACTTTTACCAAGGCATTAGCGGAGAAAGAGCTT |
| | AGAACGACTGCTGGGGACACACCAGTTGGGATTTT |
| | CAGACCAGCTATAGTGACATCAACAGCGAACGAAC |
| | CCCTCAAATGTTGGTTGGACAATATGTACGGACCG |
| | AATGGTGTGGCAGCCGGCAGTGTGACAGGTATTTT |
| | AAGAATCCTCCAGTGTGACCAAGATGTGACAGCAG |
| | AAATAGTGCCAGTTGATTACGTCGTCAACTGTCTG |
| | ATGGTCGCAGCAGCCAGTGTGCACGCCACTTATAA |
| | AACTAGTCCTCCACCAGAACCGCCTATCTTCAACT |
| | ACGTAAGCTCCGTCGAGAACAGGATAACATGGGGT |
| | GATTACATGACACAAAATATGGCAAGAGTAGACAG |
| | AAATCCATTCTCAAACGCTATATGGTACATATCAT |
| | TAACGCTGACTAAATCTGCATTCATGTATAAATTC |
| | TACATGGTATTTCTTCATTTGCTACCAGCTCTTTT |
| | AATGGACGGACTAGCCACCTGCTTAGGGAGGAAAC |
| | CAAAGATGCTCAAAGTTTACCGTAAGATACACAAA |
| | TTCTCTTCGGTGTTATCATATTTTTGTACGCGAGA |
| | AATAATATTCTGCAATAAGCGATCGAGGGAATTGT |
| | GGCATGACACATCGGAAGCGGATCGAAAGATATTC |
| | CCATTCAGCATGGCGAATGTTGATTGGAATGAGTA |
| | TTTTGATCATTACCTCGCCGGAATAAGGAGGTATC |
| | TGTTTAAAGAGAGCGATGCGACATTACCACAAGCC |
| | AGAATCAAATGGAAAAGATTGTACTATCTACATCA |
| | GGTAGTAAGAATAATTTTCTTCTGTTTCGCCATTT |
| | ACGCTCTCTGGTCGATGACATCATTAATATTTTAA |
| SEQ ID | ATGGCTTGGAGCACCGGTGGGACTAATGCCCACGA |
| NO: 24 | ATATGTCCCTGTTGCGGACTTTTATGCGGACAAAT |
| | CCATCTTCGTGACCGGTGGAACGGGGTTTATGGGA |
| | AAAGTACTAGTCGAAAAATTATTAAGGAGTTGTCC |
| | GAAAATCAAGAAGATCTATCTACTAATGCGACCGA |
| | AGCGCGGACAAGATGTCGCGTCGCGTCTCACTGAG |
| | CTAACGCAGTCACCGCTCTTCGAGACGTTGCGGAA |
| | AGACAGACCCCAAGAGTTGAAGAAGATTGTACCGA |
| | TCGTTGGTGATATCACTGAACCAGAATTGGGCATC |
| | AGTTCCGCGGATCAGGCCATGCTCTGTCAAAAGGT |
| | GTCAGTTGTATTTCATTCGGCGGCCACAGTAAAGT |
| | TTGACGAGAAACTGAAGCTCTCGGTCACCATCAAT |
| | ATGCTGGGCACGCAGCAGCTGGTGCAATTGTGTCA |
| | TCGTATGCTCGGTTTGGAGGCTCTAGTTCATGTTT |
| | CGACCGCATACTGTAACTGTGAGAGGGAACGTGTT |
| | GAGGAAACAGTATACGCGCCGCCCGCGCATCCTGA |
| | GCACGTGGTTACGTTAGTGCAGACTTTACCAGACG |
| | AGCTGGTAGACAGAATTACACCAGACTTAGTGGGC |
| | GACCGGCCAAATACTTACACATTCACAAAGGCTTT |
| | GGCAGAAGATATGCTTATAAAGGAATGTGGCAACT |
| | TACCCGTAGCGATCGTGAGGCCATCTATCGTATTG |
| | TCATCTCTAAGAGAGCCCGTTAAGGGTTGGGTGGA |
| | CAACTGGAACGGTCCCAACGGTATAATAGCTGCAG |
| | TCGGCAAAGGAATATTCCGTACGATGCTTGGCACA |
| | GGGTCGAGGGTGGCTGATCTAGTTCCCGTCGATAC |
| | CGTCATCAACCTGATGATAGTTAGCGCGTGGAGGA |
| | CACATTCGAGACGAGGGGAAGGAGTCGTGGTATAC |
| | AACTGCTGTACCGGCCAACAGAACCCCATCACGTG |
| | GCAGCGGTTCGTCAAGACCAGCTTCAAATATATGC |
| | GGAAACATCCGTTCAGTGAAGTGCTCTGGTACCCC |
| | GGCGGTGACA |
| | TAACCAGCAATCGTCTGAAGCATAGCGCCCTCACA |
| | CTCGTTCAACATCGTGCGCCCGCCGTGGTGTTGGA |
| | TCTTGTCGCCAGAGCTTCAGGAAATAAGCCTTTGA |
| | TGATCCGTGTTCAAAATAAATTAGAAAAAGCGGCC |
| | GCGTGCTTGGAATACTTTACTACCCGCCAGTGGTC |
| | GTTTGCGGACGACAACGTTCAAGCTCTCTGCGCCG |
| | CTCTCTCGCAAGAAGACAGAAGAACGTTCGACTTC |
| | AACGTGAGGAACATCGACTGGGACGCGTATATTGA |
| | GTCTTATGTGTTGGGAATTAGACGGTTTCTGTTCA |
| | AGGAGAGCCCTGATACGCTACCCAAGTCGAGGGCG |
| | TTGATACGAAGATTACACATAGTTCACGTCTTAAC |
| | CCAAGTGGCGACCGTGTTTTTCTTATGGAGATTTT |
| | TATTTTCCCGTTCCACCGCACTTCGCAATATATGG |
| | CGCTACATACTCGATTTATTAACAAGAGCCGCACG |
| | TCTGCTTGCCATAGCCTGA |
| SEQ ID | ATGAAACAATATCGTGCGCCGTCTATGCTGACGGC |
| NO: 25 | GCTTTGGGTGTTGTTAATAGCGCTCCCGACAGAAA |
| | CTCAACAAGTCAACCCGATCACATCTTTCATGAAC |
| | TTCGTAAACGAAGGCACAAGGCAGCTCGACCAAGA |
| | ACCACCAGACCAAGCTCGCCTCCTTCCCGAGTATG |
| | ACTTCATCATAGTAGGCGCTGGCACAGCAGGATGC |
| | GTTATAGCCAACAGACTAACAGAAATACCAGAATG |
| | GAAAGTGTTACTAATAGAAGCAGGCGTCAACGAAA |
| | ATTATGTCATGGACATACCCCTCGTGGCCAACTAT |
| | CTACAATTCACCGAAGCCAACTGGAAGTACAAAAC |
| | TCAACCGTCGGACAGGTACTGCGCTGGTTTTGATA |
| | ACAAACAATGCCATTGGCCGCGAGGGAAAGTTGTC |
| | GGTGGTTCCAGTGTCTTGAACTACATGATATACAC |
| | GAGAGGCTCCAAACCAGATTACGATAGCTGGAAAG |
| | ATATGGGCAACGACGGCTGGGCTTGGGATGACGTC |
| | TTACCATATTTTAAGAAGATAGAAAACTTTAAAAT |
| | TCCTAAATTCAATAATTCAAAATACCACGGAAATG |
| | AAGGATTTCTCAACATCGAACACGCTCCATATCGC |
| | AGTCCAGCAAGTAAAGCGTGGGTGAAAGGAGCGCA |
| | ACAGCTCGGTTTCAAGTATGGGGACTATAATGGCG |
| | AAGATCCAGTGAGTGTATCATTTTTACAACTCTCA |
| | ATGAAAAACGGAACACGACATAGCTCTAGCCGAGC |
| | ATACTTACATCCTATAAACGGTAGGAAAAATCTTC |
| | ACATATCAAAAGCGAGTATGGTAACAAAATTGATC |
| | TTTGATGACACTCAAACCAAAGTATTGGGCGTGGA |
| | ACTAGAAAAACATGGTATAAAATATAAAATATTAG |
| | CGAGCAAAGAAGTGATTCTGTCTGCTGGTGCAATA |
| | AATTCCCCTCAATTGTTAATGTTATCCGGAATTGG |
| | CCCAAAAGACCATTTAGAATCGAAGAAAATCAAGG |
| | TGGTCAAAGATCTTCCTGTCGGCTATAACTTAATG |
| | GATCATATAGCAGCAGGAGGGCTACAATTTGTTAT |
| | TCGGTCTCAAAACTTCAGTTTGAGCACCCAATACA |
| | TTTTAAATCACTTAGACATAGTATTTAAATGGATG |
| | AAGACCCACAATGGACCTCTGTCTATACCAGGCGG |
| | ATGCGAAGCACTTGTTTTTACAGATCTAAAAGACA |
| | AATTCAATCCAAAAGGCTGGCCAGACATGGAACTA |
| | CTTTTCATAGGTTCTAGTTTAAACGCAGATCCGCT |
| | GTTACAATATAATTTCAATTTCGACAAAGACATTT |
| | ATAGCGACACGTTTGGACCTATGGGCAACGCAAAC |
| | GCTTTTATGGTATTCCCGATGTTGATGCGACCAAA |
| | GTCAAAAGGTAGGGTAATGTTACAGAGTAGAAACC |
| | CAAAAGCACCGCCGATATTGATACCAAACTACTTT |
| | GATTATGAAGAAGATTTAAAAAAGATCGTGGAAGG |
| | AATGAAATTGGCTATAGAGATTACAAGACAACCTGC |
| | AATGAAAAAAATAGGTGCAAAATTGTATGATGTTC |
| | CAATTGAAGAATGTTTGAACTATGGACCTTTTGGG |
| | AGTGACGAATACTTCGCTTGCCACGCTCAAATGTT |
| | TACTTTCACGATATACCATCAGTGTGGAACGTGTA |
| | AGATGGGAATTGCCGAAGACCCTTCATCTGTGGTC |
| | GACTCACGTTTAAGAGTTCACGGCATTGAAAATTT |
| | AAGGGTGATAGACGCTAGCATAATGCCTGAAATAG |
| | TTGCGAGTCACACAAATGCACCAGTGTATATGATA |
| | GCGGAAAAAGGCTCAGATATGATCAAACAGGACTG |
| | GAGGAAAATGTAA |

What is claimed is:

1. A genetically modified plant having incorporated into genome thereof, a heterologous gene encoding:
a) a first fatty-acyl desaturase;
b) a second fatty-acyl desaturase; and
c) a fatty-acyl reductase;
wherein the genetically modified plant produces at least one Diatraea saccharalis pheromone precursor.

2. The genetically modified plant of claim 1, wherein the first fatty-acyl desaturase is a Δ9 desaturase and the second fatty-acyl desaturase is a Δ11 desaturase.

3. The genetically modified plant of claim 1, wherein the first fatty-acyl desaturase and the second fatty-acyl desaturase catalyze the conversion of a C₁₆ fatty-acyl-CoA to at least one mono- or di-unsaturated C₁₆ fatty-acyl-CoA.

4. The genetically modified plant of claim 3, wherein the fatty-acyl reductase catalyzes the conversion of the at least one mono- or di-unsaturated C₁₆ fatty-acyl-CoA to at least one saturated, mono-, or di-unsaturated C₁₆ fatty alcohol.

5. The genetically modified plant of claim 4, wherein the at least one saturated, mono- or di-unsaturated C₁₆ fatty alcohol is oxidized to at least one saturated, mono-, or di-unsaturated C₁₆ fatty aldehyde.

6. The genetically modified plant of claim 2, wherein the first fatty-acyl desaturase is SEQ ID NO: 4, and the second fatty-acyl is selected from SEQ ID NO: 1, and Dsac_NPAQ.

7. The genetically modified plant of claim 6, wherein the first fatty-acyl desaturase and the second fatty-acyl desaturase catalyze the conversion of a C₁₆ fatty-acyl-CoA into at least one mono- or di-unsaturated product selected from Z9:C₁₆ fatty-acyl-CoA, Z11:C₁₆ fatty-acyl-CoA, and Z9,E11:C₁₆ fatty-acyl-CoA.

8. The genetically modified plant of claim 1, wherein the fatty-acyl reductase is SEQ ID NO: 15.

9. The genetically modified plant of claim 4, wherein the at least one saturated, mono- or di-unsaturated C₁₆ fatty alcohol is selected from the group consisting of 1-hexadecanol, (Z)-9-hexadecanol, (Z)-11-hexadecanol, and (Z,E)-9,11-hexadecadienol.

10. The genetically modified plant of claim 1, wherein the plant is Nicotiana benthamiana or Camelina sativa.

11. A genetically modified microorganism having incorporated into genome thereof, a heterologous gene encoding:
a) a first fatty-acyl desaturase;
b) a second fatty-acyl desaturase; and
c) a fatty-acyl reductase;
wherein the genetically modified microorganism produces at least one Diatraea saccharalis pheromone precursor.

12. The genetically modified microorganism of claim 11, wherein the first fatty-acyl desaturase is a Δ9 desaturase and the second fatty-acyl desaturase is a Δ11 desaturase.

13. The genetically modified microorganism of claim 11, wherein the first fatty-acyl desaturase and the second fatty-acyl desaturase catalyze the conversion of a C₁₆ fatty-acyl-CoA to at least one mono- or di-unsaturated C₁₆ fatty-acyl-CoA.

14. The genetically modified microorganism of claim 13, wherein the fatty-acyl reductase catalyzes the conversion of the at least one mono- or di-unsaturated C₁₆ fatty-acyl-CoA into at least one saturated, mono-, or di-unsaturated C₁₆ fatty alcohol.

15. The genetically modified microorganism of claim 14, wherein the at least one saturated, mono- or di-unsaturated C₁₆ fatty alcohol is oxidized to at least one saturated, mono-, or di-unsaturated C₁₆ fatty aldehyde.

16. The genetically modified microorganism of claim 12, wherein the first fatty-acyl desaturase is SEQ ID NO: 4, and the second fatty-acyl is selected from SEQ ID NO: 1, and Dsac_NPAQ.

17. The genetically modified microorganism of claim 16, wherein the first fatty-acyl desaturase and the second fatty-acyl desaturase catalyze the conversion of a C₁₆ fatty-acyl-CoA into at least one mono- or di-unsaturated product selected from Z9:C₁₆ fatty-acyl-CoA, Z11:C₁₆ fatty-acyl-CoA, and Z9,E11:C₁₆ fatty-acyl-CoA.

18. The genetically modified microorganism of claim 11, wherein the fatty-acyl reductase is SEQ ID NO: 15.

19. The genetically modified microorganism of claim 14, wherein the at least one saturated, mono- or di-unsaturated C₁₆ fatty alcohol is selected from the group consisting of 1-hexadecanol, (Z)-9-hexadecanol, (Z)-11-hexadecanol, and (Z,E)-9,11-hexadecadienol.

20. The genetically modified microorganism of claim 1, wherein the microorganism is a yeast.

21. The genetically modified microorganism of claim 20, wherein the yeast is Saccharomyces cerevisiae.

22. A method of producing Diatraea saccharalis pheromone precursors, said method comprising:
a) selecting a plant or a microorganism to be genetically modified;
b) incorporating into the genome thereof, a heterologous gene encoding a first fatty-acyl desaturase, a second fatty-acyl desaturase, and a fatty-acyl reductase to obtain a genetically modified plant or a genetically modified microorganism; and
c) producing Diatraea saccharalis pheromone precursors from the genetically modified plant or the genetically modified microorganism.

23. The method of claim 22 comprising catalyzing, by the first and the second fatty-acyl desaturases, conversion of a C₁₆ fatty-acyl-CoA to at least one mono- or di-unsaturated C₁₆ fatty-acyl-CoA.

24. The method of claim 23 comprising catalyzing, by the fatty-acyl reductase, conversion of at least one mono- or di-unsaturated C₁₆ fatty-acyl-CoA into at least one Diatraea saccharalis pheromone precursor.

25. The method of claim 24 comprising oxidizing the at least one Diatraea saccharalis pheromone precursor to at least one Diatraea saccharalis pheromone.


SEQUENCE LISTING (25 sequences)

SEQ ID NO: 1 - RNA, 1035 nt, Diatraea saccharalis
  source 1..1035: mol_type = other RNA
atggctccgaatttgataaaaaacgaaggaacctttgatgtggatataaaaccagacgaactgcagctgcctaaattttccaatattaaacctaaaatattgcatgttaatttgatatatttcctttactggcatttggcagcgccatatggattatatctatgttgtacatcagctaaatgggcaacaataatatttggtttcgtcatgttcctggtagcagaactgggcataacggccggtgcacacagactctggtcccacaagagttacaaggcgaaattgccactacaaataattctaatgttattcaactcgatagcgttccaaaattccataatacattgggccagagaccacagacttcaccataaatattctgataccgaggcagacccttataattcgtctaggggatttttttattcgcactttggatggttgattactgagaaaaataaagaggttttaaatagagcgaagagcatagatgcatcggatttgtataacaacccggtactgcagttccagaaaaaatacgcagtaccagtttttgcatcgttatgtttcatattgccgacgttaatacccatgtatctatggggagaatctttcaacaattcctggcatgtcaacctgctgagatacattatgaatttgaatgttacacttcttgttaacagcgctgcccatagatggggttataagccatacgaaaaaaacattaatccaacacaaaatgtctacgtttcttttgtcacactcggcgaaggattccataattaccaccatatttttccatgggattacaaaacggcagagctcggtaacaacaagttaaatgttaccacttggtttataaatttctttgcgaatattggctgggcttatgatctcaaaactgtgtcaaatgaaatggcgagagcaagagcgatgagaactggtgatggaaaagatttatggggctgggatgagaaagatgtaactgataaggataaagaacacacagatattttaaacggtaaaacggattga

SEQ ID NO: 2 - RNA, 748 nt, Diatraea saccharalis
  source 1..748: mol_type = other RNA
actacaaataatcctaatgttattcaactcgatagcgttccaaaattccataatacattgggccagagaccacagacttcaccataaatattctgataccgaggcagacccttataattcgtctaggggatttttttattcgcactttggatggttgattactgagaaaaataaagaggttttaaatagagcgaagagcatagatgcatcggatttgtataacaacccggtactgcagttccagaaaaaatacgcagtaccagtttttgcatcgttatgtttcatattgccgacgttaatacccatgtatctatggggagaatctttcaagaattcctggtatgtcaacctgctgagatacgttataaatttgaacgttacatttcttgttaacagcgctgcccataaatggggttataagccatacgaaaaaaacattaatccagcacaaaatgtctacgtttcttttgtcacactcggcgaaggattccataattaccaccatatttttccatgggattacaaaacggcagagctcggtaacaacaagttaaatgttaccacttggtttataaatttctttgcgaatattggctgggcttatgatctcaaaactgtgtcaaatgaaatggcgagagcaagagcgatgagaactggtgatggaaaagatttatggggctgggatgagaaagatgtaactgataaggataaagaacacacagatattttaaacggtaaaacggattga

SEQ ID NO: 3 - RNA, 690 nt, Diatraea saccharalis
  source 1..690: mol_type = other RNA
atggctccgaatttgataaaaaacgaaggaacctttgatgtggatataaaaccagacgaactgcagctgcctaaattttccaatattaaacctaaaatattgcatgttaatttgatatatttcctttactggcatttggcagcgccatatggattatatctatgttgtacatcagctaaatgggcaacaataatatttggtttcgtcatgttcctggtagcagaactgggcataacggccggtgcacacagactctggtcccacaagagttacaaggcgaaattgccactacaaataattctaatgttattcaactcgatagcgttccaaaactccataatacattgggccagagaccacagacttcaccacaaatattctgataccgatgctgaccctcataatgcgtctagaggttttttttattcgcacgtcggatggttgattactaagaaaaataaggaggttttaaaaagaggaaagaacatagatgcatcggatttgtataacaatccagtactgcagttccagagaaaatacgcagtaccagtttttgcatccttctgtttcatattgcctacgttaatacccatgtatctatggggagaatctttcaagaattcctggtatgtcaacctgctgagatacgttataaatttgaacgttacatttcttgttaacagcgctgcc

SEQ ID NO: 4 - RNA, 1059 nt, Diatraea saccharalis
  source 1..1059: mol_type = other RNA
atggctcctaatgcaacagacgtcaatggcgtattattcgaagatgatgccgctactccggatatggcgttgccaaatctacctgtacaaaaagccgataactatcccaaaaaatttgtatggagaaacatcatagcattcgcatacttgcatctcgccgcaatctatggaggatatctattcttgttttctgcaaaatggcaaactgatatatttgcttacattttatatgtcatgtcaggactaggaattactgctggagcccacagactttgggcacataagtcttataaggctaaatggccactgagacttattctagttttcttcaacactctggcttttcaggactctgcaatagattggtcacgtgaccatagaatgcatcacaagtattcagaaaccgatgctgatccacacaatgcaacccgcgggtttttcttctcccatattggctggctgcttgttaggaagcatcctgaactcaagaaaaagggcaaggggcttgatctaagtgatctctatgcggacccaatacttcgtttccagaaaaagtactacctaattttaatgcccctctgttgctttgtcatgccaacagtgataccagtatatttctggggtgagacttggacgaatgcattctttgtagctgccctgttccgttacacattcattcttaatgtcacatggcttgtcaactctgctgcccacaaatggggtcacaaaccctatgataagaacattaagccttctgaaaatctctctgttgcaatttttgctcttggtgaaggcttccacaactaccaccatacattcccctgggattacaaaacggctgaactaggaaataatagactaaatttcacaactaactttattaactttttcgcaaaaatcggctgggcttatgatatgaaaacagtttctgatgaaattgtacagaagagggttaatcgtacaggagatggtagccatcacctttggggatggggtgataaagaccagcccaaagaagaagtggatgcagctatcaggatcaatccaaaagatgattaa

SEQ ID NO: 5 - RNA, 1371 nt, Diatraea saccharalis
  source 1..1371: mol_type = other RNA
atggctccagactcagaaaaaagacaaatcagttttccgcagctctactacccagcggatagggacacacttcccaaattaccttcgcaatggttgagtggtaaaaaagtacatgatggcgccgaagacctgtggaggatatacgatggcctctacgatttgactacatttatatcaacacatcctggaggtcctttttggctgacttgtacgaaaggaacagatgtaacagaagtatttgaaacacatcatcttaaaggaatcgccgaacaactgctgcctaaatattttgtaagaaaagcggcgacacctagaaattctccatttacttttgaaagcgatggtttctataaaaccctgaaatccaaagttatggagaatttacatatcatacccaaggaggaaaggaaaaagagtgacactatagtcacctatttactaatggcatttattattttaagtccactaagttgttgggtgacgcctgacaatttacttttaggagcaacattaatatttttaaatggttatgtcttatctgcattagtttgctgcggccataactattctcatagggcggatagttggcgaatgtatttgatgaacctcagtgggatgggtaatgatgaatggcggatatcgcatgttctgtcacatcacacacataccaatactgtaaacgacgtggaactgagtatactagaaccatttcttcaatatcttccgttaagagacaagcctatttgggcacaaatgggagcattctactggcctgttatatactcatttgcctttttaaatggacttattaaagagactacggcagcgattacaaaatttgaaggcaaatctttgacgtgggtgtatattatacctttttttcttcctacatggatgtggttagctggtgggttgccgttactttggactctggctatctggcaagtcacgaatgtggtggcgagtttctttttcgtatttttcggcttgaccgctggtcatcatgcacatacaaatttcttcgagggagacacaccaaggaaggaatatttggattggggcatacatcaattggatacaatagttgagagaatagattacgcaacggatcattttaaagcactcacacgttttggtgatcacggtcttcaccacatgttcccgacattggaccacgcagagctaaaatacttatatccaatttttatagaccactgtaagaaatatgagactgaacttcgtatgaccacgttttatcaatcagtcatcagtaacgttaagcaaataattagaaaaagaccccaagatttgaggaatacatcgcggcgatcaaacggcattgaaaataatttctttatgtataaaaaatag

SEQ ID NO: 6 - RNA, 975 nt, Diatraea saccharalis
  source 1..975: mol_type = other RNA
atgtccgaagaaacgaaaaataatcagcttagcaaagcgagggaagccgactggccagctgtacttttcttcattcacgtccatctgttgtcgggttacggcctttggcttttattctatgaagtaaaatggatgactttaatatttcttatagtattgactttggtagcgatgttgggagtcactgcgggggcgcatcgtttgtgggcacaccattcttatcaagccagcacaggactacgaatcattctaatgatcttccagacactagccggtcagggttcgatatacaattgggttcgacaccatcggcttcatcatgcacatttcggaacagacggtgatccatatgactataaaagagggttcgtacaatcgcatgtggtctcacgcctccgtcgactcagtccttatcagtctcaactgattagtgaaatcgacatgtctgacttggagaatgatcctgttatcatggtgcaagataaattgtattgggtgttatactccattatatttcttcttctaccactaaatgcaccgcttgaatactggagcgacacagtgctcagttcggtattcgttattggctttcttcgttacggactggtgttgcacgcatcgtggcttgttgaaagtagtatctgcgtttggggtttgaagcctggtgagaaatgcccacctgactctaactggattttcgtgctgtcaaagtcgttatggccccattaccattatttgatcccatacgactataagtctggagagtacggaacatacgattcaggatgcacatcagcattcattcgagtgttcgctgcattgggcctagctacaaatctgaaaacagtagatactgccacagtacagaaggctcttgctcactccgcccgatcgaaactcccagttcagacgtgcttggaggaagctatatcaaaacaaattctacacgaagaacattacttgcgtagagggtga

SEQ ID NO: 7 - RNA, 966 nt, Diatraea saccharalis
  source 1..966: mol_type = other RNA
atgggtgccaaagtgtctaggactgatttcgagtgggtttacaccgaggaaccacacgctagccgccggaaaatcatcttggaaaaatacccggaaataaagcagctctttggctacgaccctctattcaaatgggtggtgactgctatggtgatcactcagttgatcaccttgcctatagtgcaacacctctcctggccgatgaccatgctgatggcttactgcttcggtggagtgatcaaccattccctcatgttagccatacacgaaatagctcacaacctggcgtttggtcacaatcgaccatttcacaaccggctctttggtttcttcgcgaatattccaatcggcataccgatttcaataagctttaagaaataccacctggagcaccataggtatcaaggtgatgaagtgattgatacagatctacctacacttatagaagctaaattgttttgcaccaccggtggcaagctgctgtggctgttcctccagcctttcttttacgctgtcagacctctggtggttagacctaaaccgccgacacctctggaactgatcaacctggtgatacagttgttcttcgacgccatagttgtgaaattgttcggttggaaggtgttggcttatttagtacttggatcgttaatggcgatgggtgtacaccctgtagctggtcattttgtctccgaacattatatgttcaagaagggcttcgagacatactcgtattatggacccctgaattggataactttcaacgttggctaccataatgaacaccatgacttcccagcggtgcccggacggagattaccagaggtgaaacgcatagccgctgaattctacgacaatctgccgcaacacaacagttggtccagtgtcctatacgatttcgtgactgaccccgacattggcccgtacgctaggatcaaacgcaaacatgtaggcttagatagctag

SEQ ID NO: 8 - RNA, 1128 nt, Diatraea saccharalis
  source 1..1128: mol_type = other RNA
atggctccagcgcaacaggacgttatgctgtgtaaagaatcggtgcagtcaaaaataaaaataagacatatagatagtgaatataaacgtgaaaacggtgatgtcagtgaaaataacaacgttgtggtgaaaaatggtgccagtgatgaaacaaccagtgataaagacttcgatttgagcaaatatgaagcgatgaagttcactgcacgaataaagtggccagaccttatagttcaattgtcattacatttagtttcattatatggactttatctaattttaacatttgaagtgaaaatcctcacaattttatttgtgttagcaacaatatacacgtcggggttcggcattacggccggtgtacacagactatggtcgcatcgagcgtaccgagctaagctgccgctgcggatactcctcgccttcctattcacaataactggacagcgcgacatatatacctgggctctagatcaccgggtacaccacaagtattcggagacggtggctgatcctcacgacgtcagacgcggcttctggttcgcgcacgtcggttggctagtactgactccccatcccgctgtggagaaccgccgcgccgcattaaagctcactgctacagatctcatcgccgatcctgtcgtcaggatacagaagataactttcataccacttttcgcacttctgaacatagtggtgcctacagcggtgccgtggtactgctggcaagagaccctgttcaatagctttgtgctcagttttgtcactcgattcaccatcaccttaaatatagcttactgtgtcaacagcttcgcacatctgtggggcaacaagccttatgatagatttatcaaatctgtggagaacaaagcggtgagtctggcagccttaggtgagggttggcacaactaccatcacgtgttcccctgggactatcgaacctctgaactggggaggatcaacatctccaccaacttcatcgatgccttcgcaaaaattggctgggcatatgatctcaaagctgcaagtaacacaatgattatcaacagagctaagaaatcaggagatggaacgtttggagaaaccgaagagccagatccgaatccttcagaatataaacacttgtag

SEQ ID NO: 9 - RNA, 1359 nt, Diatraea saccharalis
  source 1..1359: mol_type = other RNA
atggcaccaaaaaatttggaatatttagaaatcgctcagaagagagcctttgagaagaaaactcatgttagttttcctcaacttaaatatccctccctcagagatacaggatttagagatccagtgcaatggctgactggaaaagctttggacgatggagcagaaggcttgtggagaattcataattctatttatgatcttactagtttcatcgataaccatcccggtggtgcagaatggctagaactcacaaagggaactgatataacagaggcatttgaagctcatcatcttaaccctgtagtagaaaagatgttagataaatattttgtaaaaaatgcaacaacgccaagaaactccccttacacgttcaatgaagatggtttctaccgtacacttaaaaattctgtgagagaagaattgaaaaaagtaccaaagaacattcctaaccatgcagacagaatcattgatggattatttattacatgtttggcaacagccgcattgacttgttgggcgaacaattattgggttgtgatgttatcatatctaatctcttctttaacattagcatggactgttgttgcatcacataactatatacacaggaaaaccaattggagaatgtacatttttagtatgtgcttatggtcatacagagatttcagggtgtcccatgctctctcacatcacttatatccaaacactctaatggatttagagataagtgcatttgagccaattatacaatggaatccgcggccagacaagccgtttttcacatactttacttttttgtttcaatgtttattatatccctttgccttcatgatgagttttgtgaaaagatttcttctcaactttgtaagaaaagattttttcaaaaaccattatcgctggcatgatgtaatcggtcttcttcttcccgtttggatgtgggtcgtcagcggatgttccttcagtgacgctattatcaactggttatggatcaattgcactagcagttttatcttcatgatgatcggtaccaatgcagcacatcaccacccacgaatattcaaagatggtgacgaagtcaggacaattacacctgactggggaatgcaccaacttgaggccgtgatggatcgtaatgatatcaacgggagccttttcaaagttatgaccttcttcggtgatcacgctcttcatcatcttttcccaactctagatcatgccgtgttaccatatttgtatcctgtatttttggaacactgtgaaaaattcaaagcaaacttcagacttacgtcacaatttgatctttttattggtcaaatcaagagggcaaatgaaaagcaattcaaactgttagatgattaa

SEQ ID NO: 10 - RNA, 1122 nt, Diatraea saccharalis
  source 1..1122: mol_type = other RNA
atgacctcttctttgcttctggcgagcacgatcttcacaaaagatatcaaacaagtagaagatgacatgccgcaaatttctacacgtctgtcgagcgggaagaacagagattatgaatggcagatagtatggagaaacgtattagggttcgtctatctccacgcggcagcattttatgcaatttatttaacaataacaggcagattcaaattatatacttacatatttgctttaacttttgctatactcgctgctatgggagtaacggcgggagcacataggctatgggctcatcaggcgtacaaagcaaaatggccactaagactgttcctagccgttttgcaaactatggccttccagaatcacatttacgaatgggtgcgagatcatagagttcatcataaattcacggaaactgacgctgatccccacaacgccaagcgtggtttcttcttctcccatatcggctggctgatgattcgcaaacataaagacgtgttcgagaagggtatcaccattgacatgtccgacttggagaaagatcctatcgtcatgtttcagaaaagaacatatttgttcgtgatgccacttctctgcttcataatacccgcatggatccccatttacttctggaacgaggatccttggacatcgtggtatattgcagctatatggcgttatacactatcccttcactttacttggctcgtcaattccgctgctcacatatggggaaataaaccatacgataaatatataggcgcaactgacaataaaactgttgctatttgcgcatttggtgagggttggcataactaccaccacgtgttcccatgggactacaaagcggcagaactgggaaattattcaacaaacatgtcaacggcgttaattgatcttgctgcaaattggggtttggcttatgatctgaagacggtgtctgtagaaatgataaagaaacgtgcagcaagaacaggggacggcactcatccatctaataaagacaccaatgcatctcacgaagatcatcaccatccagaaaatcctgtctggggctgggaagataaagacatgccagaagaagatagaagatacgcagaaattcataaaaaagtagaatga

SEQ ID NO: 11 - RNA, 1353 nt, Diatraea saccharalis
  source 1..1353: mol_type = other RNA
atggcgcctaaagatcccgaatgggaaagtgaagctctaaaaaggatagatgagaaaagaactcatgtcagtttcccaaagttaaaatatccgtctttaagggatgatgctatgagggatcctatacaatggcttacaggaaaagctatggatgatggtgcagaaggtatatggcgtattcatgataagttgtatgattttacaagtttcatgaagcgacatcccggcggcgaagaatggttggagttgacgcagggcactgacataacagaagcgttcgaggcccatcacataaatccgacaacacaaaaactcttagacaaattctacgtcagagacgcaaacacaccaaggaactcccctttcacgtttaaggaggatggtttttatagaactctgaaaaaggcggtgtacaaagaattggagaaaatacccaaagatgtatctaaagctgcggataggataacagattggctatttgtatctcttttatgtagctccgcaatggcagtctgggtggaaaatatctatgctgcgaccgtttggtacatttttgcatccgtaaatctagcatttttgacggtggcatgtcacaactatattcataggcgtacgaattggaggatgtatttgtttaatatgagcatgtggtcttacagagatttccgtgtgtcccatgtgatgtcacaccatctttacacgaacactttaatggacttggagatcagtgcactggagccagttttgttcttcaatccaaggaaagataagccgatgtacgcacgactgggtttcataactgaattgttcttctttccgttcgttttcctcataaattttatgaaaagattcatatcagtgttcatcagaaaaggatttttcaaacgacattatcgatggcatgatatgataggatttctcctgccatttattatgtgggtcgcaagtggtgcgtcaatccttcacgttctgtattattggctttggatcaactgtacaggaagtttaatattttattgtataggcgtaaatgcggcacatcatcaccctgaagctatcaaagatggtgataaaccaaggagtgaaacaccagattggggtgaacaccaagtggaagctctcttagaccgcaaagatgtgaataacaacgtcttcgccgtggtcgtgttattcggggagcatgccctacaccatatgtttccgacactagatcatgcggtgttgaaatatttacaccccatcttcatcgaacattgcgagaaattcaaggcaaattacagagtatcgacgcagttcctaatggttttgggacagattaaggaatgtatgcgtgcagaatttaggattgtttga

SEQ ID NO: 12 - RNA, 1056 nt, Diatraea saccharalis
  source 1..1056: mol_type = other RNA
atgccgccacaaggacaagagcaagattcctgggtgctaaacgaaactgatgtcaaagaccaagatgtcaatcatctggtgcctccatcagctgagaaaaggaaaatggatattgtatggaggaatgtgatcatcttcgcctacctccacttaagcgccatatatggtgcataccttttcttcacaactgtcatgtggaagactatgctcgctacttatatcctgtacgtcatgtcgggcctcgggatcacagccggcgcgcacagactttgggcacacaaatcctacaaggcaaagctaccattgcgtatcttattaactttcttcaacacaatggcttttcaggattcagttttggattgggctagagaccataggatgcaccataagtattctgaaactgatgctgatccccacaatgcgactagaggtttcttcttctcgcacgtcggttggctgctcgtaaggaaacatcctcagatcaaggctaaaggaaacaccatcgacttgagtgatctgtgggctgacccagttcttcgattccagaaaaagtactacatgtatctcatgccattggcttgtttcgtcataccaacagtgttacctaccctctggggtgagagcctgtggaatgcttacttctgcactgctatcttccgctacgttttcgtcctaaacagtacctggctcgtcaattctgcagctcatttatggggtgaaaaaccttacgatagacatataaatcctgtagaaattaagactgtttctatagcagctttaggagaaggatttcataattaccatcacacattcccatgggattataaaactgcagaactggggaactactctttcaattacactaagctttttattgatacgatggctaaaattggctgggcttatgatctgaaaactgtttcacatgaagtgatagagaaacgggtaaaaagaaccggtgatggaagtcatagtgtgtggggttggggtgataaaaatatacccgaagaagataaaaatgatactacgctcataaatccagccaagtctgaataa

SEQ ID NO: 13 - RNA, 1077 nt, Diatraea saccharalis
  source 1..1077: mol_type = other RNA
atgacacctcaagtagacccgatacccagtggggttttgttcgagacagagacacaaacggcggacttaggactagacgccgatgtctcaaaactgaagaacgctgccccaaagaaatatgaatacgtctatttcaacatagtttggttcatatttctccacaccgtttcgctgtacggattatacctggcattcacatcggctaaatggcaaacgaacctctttgcatccgtgatgcacttagcgtgtgctatcggagtaggcgctggatctcatcggctctggactcacagaggatacaaagctcgcacgcctcttaggattttacttatgatctggcagacaatggcatttcaggactgtatatttgaatgggcacgcgaccaccgcacccaccacaagtacgctgacacagatgctgatcctcataactcggtgaggggtctgtttttctctcactgcggctggttatgttgcaagaaaagccctgaggttatcgaaggtgggaagaggattgacgtaaccgatctatacgaagatcctgtcgtcatgtttcagaagaaacactacatgaagatgatgccaatcttgtgcttcgtgttgcctaccgtcattcccgtgtatttctggggtgaaacctggctgaacgcattctgcataccaacaatcctacgttacacgttcggtatcaacgttgtgtggtctattaatagtttcgcccaccactacggcttccgtccttatgacaaatctctaaacccacgtgacaatgtcgccatctggatgttctgcttagagggtttccacaactaccaccacacctttccttgggactacagagcgacggaatacccattttacaacctgctcacacccacggtcgtcttcatagatttaatggctaagataggtcaagcatatgatttgaagagtgtaacgcctgatattataaaacaacgagctcttcgcacaggagacggcacccacaacctctggggctgggatgatcccgagtttactgaaaaattgaaagcacagtacagtgtcataactaatgtcgaaaaaaaatttgattaa

SEQ ID NO: 14 - RNA, 1059 nt, Diatraea saccharalis
  source 1..1059: mol_type = other RNA
atgcctccccaaggccagtccccctcatgggtgctggaggagtctgacgccgccaccgacgacaaggatgtggcgaccccggtccccccctcggctgagaagaggaagcttcagatagtgtggaggaatgtcaccctcttcgcgttcctccacgtcggcgccttatacggtggttacctgttctttacacgggctatgtggaccaccagaatcttcactgtgattctgtacgtcatgtccggcctgggcataacggcgggtgcgcacaggctctgggcccacaagtcgtataaggctcggctgcctttgagaatactactaacattgttcaacacgctcgcgtttcaggactccgtgctagactgggctcgggaccacagaatgcaccacaagtattctgagaccgacgccgacccacacaatgcaacacgtggcttctttttctctcacgtcggctggctgctggtgcggaagcatccgcagatcaaggctaaggggcacaccatcgacatgagcgacctctgtgcagatcctgttctgaggttccagaaaaaatattacttgactttgatgcctctcgtttgcttcatcctgccaacctacatcccaacgctgtggggggagtcactttggaacgcctacttcgtctccgcgatcttccgctactgctacgtcctgaacgtcacctggctggtcaactcagccgcccacaagtggggagaccggccttatgacaagaacatcaatccggtagagaccaaaccagtctccctggtcgtgtttggggaaggtttccacaactaccaccacacgttcccgtgggactacaagacagctgaactgggagactattccttgaacctttccaagctctttatagataccatggctaagattggatgggcctatgatctgaaatcggtttccaaggacattgtagagaaacgagtaaagaggacgggagacggtagccacgccgtgtgggggtgggacgacaaggacgttcccgtggaacagaagacagcagcggtaataatcaaccctgaaaagactgaatga

SEQ ID NO: 15 - RNA, 1422 nt, Diatraea saccharalis
  source 1..1422: mol_type = other RNA
atggaagaaattgaaaacgtaaacgaaatcctagtgaaatcacaattgaattcgcctgtagtgaacttctattctggtagatcagtatttattacaggctgtaccggattcttaggcacggttttacttgaaaaactgctgtttacatgcggacataatctaaaacacgtttacatactaataaaacaaaaggatgaccagaaaattgatgaaaaggtgtcaaatttctttaactcccgtgtgttccagaggttgaacaagcacaatcccaacttcagagcgaagattataccaataagtggtgatctttcgaaaaaatgtattggtatcaatgaaaacaacttatctttgctccgcaaagaggtatccgtagtgtttcattctgccgcagacatttcttttgatatgagtgtgagtgacgctgtcaatataaacactaaagctacagaaaaattgttgaaaatttgcaaagagatgcatcagttaaaagcattcgtttacgtatcaactgcttacagtaactgtaacagaaatgttatagacgaaaaagtataccctagtgatgtaccattagaaacactatacgaagtactcgagcattgtaaaagtgaaagattgactcaatatttgctcaatggaagacctaacgcctactcatattcaaaggcactttccgaagaactaatacagaattacgcgaattctgtaccatcaatcatcatccgtccttcaattattatatcatcggtgaaggatccgatgcccggttggttgggtggatggaatggcctcagtcgagtgatacacgcggggatgaacggagatatgaaatgttggatcgcggaccctttgtgtactacagacatcattcctgtggactacacagcgaatatcatgatagtttcagcatgggaagcaggagaaaaattgaaaagtagacatcagttaaaagtatataactgttgctcgggtctgcagaacccaattaattctggaaccatagtgaaagattgtttggaatacaacgaagagtatgagaaagataaatctaaggttaacaagatatttataatgcggaataacttttacgtatatttaatgttcctattccttcatattatacctgctttcgtaattgatgtgttctactttctaacaggaagaaaaatgataatgttaaaaaggctgaagaaaataaaactgttgtcaaaaatgttcaaatcattttgtgtcaaagaattcttattcattgacaataatgttcgtaagctgtatgaaagcttaaatgaatccgagaagattttgttcgatttcaacgtcaaaaatttacaatggaaggagtacatgaagagctttgtgattgcgatgaaagaatatagtaaggagactaaaatcagcaaaaaacaaaacaaaattgaataa

SEQ ID NO: 16 - RNA, 1533 nt, Diatraea saccharalis
  source 1..1533: mol_type = other RNA
atgccggatgaatcgcaagtgcgtgccttttactctggcaagaatttcttcataactggcggtacagggtttgtgggattatgtctaatagagaagatattgaggacaatcccagacgtcggcaaactctacttgctaatgaggccgaaaaaaggaaaagacatcgttgaaagactggacgagttccctaaacacccgatatttgagaaactaatagaacaaaagacaattgacatatttcacaaactagtagccgtcgctggggatgttggtgaagaggatctcggtctcagtccggaagacaggaaaattcttgttgaaaatgtgaatgtagtggtacattccgctgccacacttgatttccaagacaatttaagaccaacggtacagatcaatctactgggcactagacaagttatggaactatgcaaacaaatcaaaaacttgaaggtcatgatccatgtgtcttcagcttacgtcaattcatatttaacagaagcacacgaaaaagtatatgaagctccagatgatgcggaaaaaattatatctttagttactacactctctgatgaggctttagatgaaatagaaccaaagatattaaaggatcatccgaatacatacacattcaccaaacatcttgccgaacatgaagtaaagaaatgttctgacttgttcccatgtactatagtcagacctactatgattgtagggtcatggcaggaaccagtgcctggttggacatgttccaaagttggtccgcaagggttccttatgggtgcgagtaaaggtgtagtcagaagattacctctctcaaaagacaacatcgctgactatattccggtggatgtggttgtgaacgaattactggtggctggatggcatgccgctaaatctaagtctggtttgactgtttatcactgttcctcgtctacttgtagaccgttccgttggttcagtatagagtattctctcaatgataagttacatgcatatccactgaagagtgcagtatggtatccatatcttgcatttaattcttctttgttgagttttcgtctgtcggccatatttatacatttcttcccagcaattttattggatttattacttaagttaactggaggtcgacccatattatggcgattgaatcgtaacgtatggaactctttagctcgtctagagaagttcatattcaccgagtggctattccataatccaaatactctagatctctgcaagcagttgaacaaaacggacagagaattattttatattgacatttcaactttggaatgggaagagtactttacgaatcttcaaaaaggcgttagaagatatttaaacaacgaaaaagaatctacgctcccagctgctaggaaaaagcaaaccaaattatacatcttccatttgatctggcaagtggccatcatttcactactctggtacttcgctgcctgcctgatgggagtcagtatgatgaaatgcgtgtgggcagtgcccggtatctacatactatactcgtgtttgtaa

SEQ ID NO: 17 - RNA, 1878 nt, Diatraea saccharalis
  source 1..1878: mol_type = other RNA
atgaaccactcactggagaattctccgctgatacccaccgacatgaccagagagaagatggagaagtgggtggcagcgcaacagaaaggagagaaaatagacattgacatttatggtaaacccacagagaaacaaattaaagagttggaaaatataaggaatttgagcagagaactacaggataacttacacgagttagaaaactcggtacgcatagctgacgtggaaaaccaagcgatgaatccaacggcacccattttggattactctgaagaccacgaattcgtgtctgccaataatttggacaattattacgctgaagaagacaaaattgacgcgaaagaagaggagaagagacggttaactaagggtaaatgtggcacatcagagattcaacagttttataaggaccaatcggtttttttgacgggtggtaccgggtttttgggtaaagtgcttattgaaaaactgctccgtacttgtggagacatcgacactatatacgttttaataagaaataagaaagggaaagatgccagagcaagattacatgaaatgttggatgaatttttgtttgagaaagcacacgaaattaatccaaagggtatacataaagtggtgcctataattggtgatatggaactaccaggccttggaatgtgcgaagaggacagaaaaactgttaccactaaagccaccatcataataaatgcagcagcaacggtgaaattcgatgagaagctttcagtgtcgactgctatcaacgtaaagggtactaaggaggttttaaaactagctaacgaatgcagaaacttaaaagccatcacacacgtgtccactgccttttcaaatactcacattaaatatgttgaagaaaaattttatgagccccctatgtctgtagaagctctggaagctgtgtcagaaataaatgatgacatacttgacgacattttgccgagtttattaggaaaacggccgaacacctactgcttcacgaaagctgtagctgaagaagctgtcaggacacacggggcaggtctacccatctgtatcgtccgaccttccataattgtgtcgacatacgaggagccagtacgtggctggacggacagcgtgtacgggcctacaggactcgtgatcggaatcggcaccggggtgctcaggactatgtatatggatctagacatcgtggctgacatggtacccgtcgatctagtggtgaactcaatattagcatcaacgtggtataccgctaagaactacaaagtgaatcaaacttcagatatccctatttacaatttcgtgtctggagcacagaatcctataaaatggggccaatttatagaactcaataggagatatggcatcgataaacccaccactaaagctgtatggtattatggtttgaatcccaccaataattactacatgttcctgttctacaatttctttttacattatctgccggctctcctcatagatatgtacagcgctctcattgggagacgaagagctatgttaaaactttactcgaaggttatgaagttggccaacattttgttttacttctcaacacaagactggaagttctctgatcgtaatgttcgctcaatgtggtcgtcactatcagaggctgacaaagcaatatatccgttcagtttaagtgagatgtcatgggagaggctttgcgagaagtttttaattggtttaagagtgtatctaataaaagacgacctttcaactctacccgaggctagaaaaaaatggaacaggttgttctaccttcaccaaatgctgaagacccttacaatagcattaatcttaaacttagcgtatctcgtattgaggccaattttcaccgccatacttggttga

SEQ ID NO: 18 - RNA, 1578 nt, Diatraea saccharalis
  source 1..1578: mol_type = other RNA
atggcgtccgagggtttctcagcgtctgaggtggatggtatgccggatcggatagcggagacgtttacaggccgtcgcttgctggtcaccggcggtacaggtttcatgggaaaggtgctcatagagaaactgttgagaaaatgtccagacattagtcagatctttcttcttgtgcgcaccaagaaaggcaagaatccgaaacagagattggaagagatattcaacggagaattgttcgaaatgctccgcaacatgagaggtggtatcgaaccactcttggagaaggtgtcgatcataagcggtgatgtgagtgcaccagacctggccatgagtgagggagacagacagaagatcatcgatgaagtcgatgttgtaatacacgctgctgctaccatcagatttgatgaagagctgaagaaggcagttcttctgaacgtacgaggcaccaaattgattttagaactcgcaaaacaatgcaaaaacctacagctcttcatgcacatatcaacagcttactgtcatctgcacgagaaactattggaagaaaagccgtatcctccacccgctgatccccaccagattattcaggcgatggaatggatggatgatgagaccatcgcaaccgtgacacctaaactcctaaataagctacccaactcgtatgccttcaccaaagctttaggcgagggtcttgtggtggaatacatgcagcacattccagccgtcatactccgaccttccatcgtgatcccgatatggcaagaaccacttcctggatggactgacaatattaacggacctactggtttgctgattggtgctggaaagggcgttatcagaactatgtactgcaagagcaacagttatgccgactatctgccagtcgatgtgttcatcaatggaatcatgatcttggcttggaactatttggtttgcggtgataaagagaggaacatagtcaacttcacgtcatcagcggagatcaaggtgacatggaatgagctgatcgaagctggcagggagatcatcatgaacagagtacctcttaatggagtagtttggtaccctggtggttctatgaagcattctcgtctgtatcacaacatctgcgcgttcttctttcactggctaccggctatatttatcgatactctactcttttgtcttggctacaaacctgttttgatgcgtgttcaccggcgcataagtaaaggtttcgaagtgttcgagtattatacaaacaaccaatgggacttcaaatctgacacagcacagaaagtgagaacgaggatgaacgctcgagagaggcgggactataaagttgatgctgttggcgtggatatttccaaatacttcgaagactgcatcaaagctgccaggatctacatcctgaaggagtacgacgacactctcccggcagccaggagacacatgagagtaatgtattgggtcgatttgattgcacaaatactcttttgggctctaatattgtactggatgaagggtctcttttctggaatgggctccttcttctttggctcgagcagtgacgtcacgccatcagtaatggcggcatag

SEQ ID NO: 19 - RNA, 1551 nt, Diatraea saccharalis
  source 1..1551: mol_type = other RNA
atgggctttttagaagatagagatctaagtgatgtgccaggcatccaggaatattttaaagggaagactatattcataacaggaggatcaggttttttaggaaaggctttgatagagaaattgctgtattcgtgttcagatttagacagaatctatatcttgatgaggtcaaagaaaggcgtcaaggctgaagatagactggctgagctttattctgctatagcattcggtcgtttgaaagcggaaaaaccggacatttttcaatcaaaagtttttgttgttaccggagatgtcatggagccaggattgggtttatcagaagaagatagaactctgttagtgaatagggtacacataattttccatgtggcggccagcgtaagatttgatgatccgttgcattatgctgctaaattaaacctcggtggtaccaaagagatcgtagagtttgccaaagatgtccgtaacttatcttctctggtgcacgtgtcgacgtcgtattcgaacacgaaccgtgacgtcatagaagaggtcatgtaccctccgcacgcggactggagggacacgttacaagtatgcgaatctatcgacgaacagtccttacgcgtgctgacccctaagtatctcggtgaattacctaacacttacacgtttactaaacagctggctgaacacgcgatttacgagaacaagggtctactcccagtcgtcatcattagaccatctatagtgatttccagtgtggatgagccttttcctggttggatagaaaatttcaatggaccagtgggtatactggttgcttgtggaaaaggtattatgcggagcttgtacactgatcctaatttgatagctgactacatacctgttgatttctctattaagagtattatagctgctgcatggattagaggtaccaaggagttagaatcaacagacgatatcccgatctacaactgctgcgctggaaatctcaataacattacaatgtatgagctcgtggatatcggcaaacgattggcggctaatatgcctcttaatgacatgttgtggaacgttggaggatcccttactacaagcaaaacgttacattatattaaggtgttgctgctacattgccttcctgccatatttgttgatgcactgttattgatactaggaaagaaaccaatgttattgaaactccaaagacgcatttacatagcaaacttagcgctccattattacataaccaagcaatggacgttcgacaataaaaacttcgtccttctccgatctcgtataaaggagcaagacaaaaagcaattcttttatgacatcgaaaacgtggataaacaagaatatttcaggaaatgttgcattggaggaaggaaatatttgctgaaagaaaaagatgaagatttgccaaaagctaaagctcattatgccagaatgttgatactggacaaatgtgtacagataatattttatggatatttagtctggtggattttgaacattggatatataaagaatttactaaaatttgtgtatgatatatttatgctttga

SEQ ID NO: 20 - RNA, 1497 nt, Diatraea saccharalis
  source 1..1497: mol_type = other RNA
atggcgccacctgttagcgttaaagactattatgcggggaaatcgattttcatcaccggctccacaggttttatgggaaaagtcttagtggataagattttaaggtgctgtccggatgtgaagaatctgtatctcctcatgagagctaaaaaggggcacagcgttaaggagagaatcgacgagtttttgaactgcagggtgtttgactatttaaaaagtgcacaaccagagcagctgcagaagctacaagtggttccaggtgacatcttagtggagaacatgggcatgtcaatggacgatcgaacaatgttgcagaaggaatgtcagatagttttccattgtgctgcatgcgttagattcgatatgtttattcgcgatgctctcaacttaaataccgttggaacccagagagtgttagatttagtctccggcatgacgaagatagaggtcttccttcacgtgtcgaccgcgtacagtcgttgcgaattagacgttttggaagagaaactgtacccctccaaccacagaccacagcacgtaatcaactgcgtctcctggatggacgatgaaatgctggaacatttacagcccaaacttatacaccctcagcctaacacgtatgcttatacaaaatcattgaccgaagatctcgtgtcacaatatgctgggaaatttccaatcatcatagcaaggccatctattgtaacctcatcatacaaagaaccaatgcctggatgggtagacaatttaaatggacctacaggcctcatgattggcgctggtaaaggtgcgatcagaactatatatatggacgagtctctccgcgccgatgttataccagtagatatagtagtcaatgcatgcatactcctcgcttattccacaggtctagaaaaatcgaaagagctgcaattttgtaatttgacactgtcagacgacaaccctctcacttggggtgcagccttggattttggtcgtaaacacgtcacagagttcccgtcctccgtctgtttgtggtatcctggcggttctgcgaagacatcctggttgcagcaccaaatagctgtgttttttacccatctcttgccggcttatttcgttgacctcctcttgatgttgtttggaaagaaaccttttatggtgaaattgcagaagagaattaactacggcttacaagtcatacaatattatacaacgaaggagtggtatttcaaaaatgactatttaaaagctatacgagagaaagtgagcgcagaagacaacaaaatattttatactgacactaaggtaatcaactgggattcatacatcagggactacattaaaggcgcacgtgaatactgtctgaaagaagacccagtgacattgccccaagctaggagacttaataggcagttatattatgccgataaatttttacaaatggtcctgtattcattattaacatacgtaatttattcatacttcaaaatgttcataagtatgatgagtgtttag

SEQ ID NO: 21 - RNA, 1566 nt, Diatraea saccharalis
  source 1..1566: mol_type = other RNA
atggcatcagaaacatcagtgagggagttctatagaggcaggagcgtcctggtgactggaggcactggtttcatgggaaaagtgctgattgagaaattgctttactccattcccgatatcggcaacatttatatactgctgcgccctaaacgtggcaagtctgttacgcagcgtttagaagacatgcaacggttaccattattcgaccgtctcaaaaccgaacggcctagtgcattcaaaaaaatgaaagcactacagggtgacgttctatttcataacttcggcctctctaaaagcgacatagagaacctgtctgccgaagtctccgtcgtattccattttgctgcaacgctgaaactagaagcacctctcaaggataacgtagatatgaacaccagcggtacccaaagaacccttaatattgcgaggcaattgaagaacttaaccatattcgtacatttgtcaactgctttctgttaccctgactacgaagtattaaacgaaaaggttcacgcgccaccagctaagccggaagatgttatgaggttaattgaatggttagacgataaacaaatatcgatacttacccccgcacttctgggtccacaccctaactgctatacttactctaagaggctcgctgaaaatatagtcgaaaacgcttttgaagaattgcctattgttatatgccgaccgagtatagtatgcccgtcttattcggaaccgctgccaggatgggtggacagcctgaacggaccagttggtttaatgttaggagctggaaaaggtgtcatcagaaccatgatgtgtgatggaagtctagtcgctcaagtaatacctgtcgatattgctatcaacgcagttatcgctgtctccaaaatcgaaggcagcaagaagaagaaacctgaagtaataccagtgtacaatttaaacatcggacatcaaaaaccaactacctggggttcagtacttcaagtagcgaaagactatggacggaaggcaccacttgattggccgttgtggtaccccaacggagatatcaccacgaatatggcattgcatgagttcagaagatacttctaccatctgattccagcttacttgatcgatttcctcatgttactattcggacagaaacgattcatggtaagaattcaagacagaatcagccaaggattgtacgtgctccaatacttcactactagaaactggtctttcggctccgaaaactacgatagcatacagaaggccttaaacgatgaagaaaaagtgatattcaacactaacattgaagatgctgatcgcgacgagtacatgcggcacagcgttaacggcggccgagtgttctgtttgaaagaagatcctaagaatataggcaggaacaagatttatcacaacgtgcttttcgttctggactgtatcgtgaaggtcctgttttggctcctgatactctccttcgtggtgtcctggtgcacaccactgaaggcaatcttctccatcggtggacccctggtcaaacctttacctttcttaggtgccgctgtcttcgacggcaatgacttttag

SEQ ID NO: 22 - RNA, 1308 nt, Diatraea canella
  source 1..1308: mol_type = other RNA
atgactttaggtgagaggccccagattccgcagttctacgcgggaagatcggtcttcatcaccggcgctactgggttcattggacaagtgctggtggagcgactgttgtatacgtgtcctgatatcgagagattattcctattgctgagggagaagaaagactctgcgcctatgcaacgtttgcgtttacttaaagattcgccagtgttcgacaatatccgccaaaaaaacccctcgcagttggacaagctgaccgcggtgtctggagacatcacgaagccgcagctcggtctccagcgagaatcaattgatctccttcagaacgtatcggtagtactccattctgctgcgacgttaaagttttcggagcccctcgctgcggcagtagaacaaaacatcaagcctgtcatcaagattatggagttgtgtgataatctacccaacatggaggctttcgtgtatgtatcaactgcgtacagtaacgccgagctgagcacggtggaagaacgcgtgtacccgccccccgtgccgctgaaggatctccttactctcgtagatacctcaacaccagaacaactggccgaagtcacacaagagtacatagcaccgaagcccaatacgtatacatattccaaggctatggcagaggcggtggtgcaggagcacaccagaaggacatattccgtagcaatattcaggccgactatagtggtgtcatcgttgcgtcacccgtatgctggatgggtgcaaggactgaacggtcccagcggcgtggtggcgagcgcgggcaagggcctactgcacgtgcagtatggacagcgcacagcgcgcgccgacatgctgccagtagacatcgccgtcgacaccctcatagccgtcgcctgggagactgccaccgacaggacgcccgaagtgagagtatacaactgcagtacttgtgagaaccctacaacttggtatcagttcgaggacggcataagacgaaatctgcgcgagcacccgttcgacaacgccttctggtggccctgcggaagcaccgtcaacaattggttattattcaaaattctggagtttctattacacacgatgccgttacatttagctgagtatgtcatgtcgttattcggagctaaatcaaggatcaacatgataacgttgagcgcgcggttacaaggcatgaattctgtgctggctttcttttccatgcgggaatggaaattcgatacacgcaacgtacaaatgttgaggagtaaacttacgccacaggacgccgccatatacaatctggatcctcacagtatagagtaa

SEQ ID NO: 23 - RNA, 1650 nt, Diatraea saccharalis
  source 1..1650: mol_type = other RNA
atggacggagtccgtagcaacgattgtaaagaaccattcaacgataagggattttgtaacgataccgctataatgaataacgatgttacattagcgaatataaataacaatagttgtaacgacagattgaacggtaatgttattgaagacggcaacactagtaatatgagtgaagtgcaaaagttttatgatgggaaaaatatattaattactggagctactgggttccttggtaaaattctaatggagaaactattgaggagttgtccgggtgtagaaaacttatatttactggtcagacagaagcgtggcaaagacatctacacgagaatagaagaaatatttgatgatcccgtgttcgatcgtctcaaagaagaagtaccaaagttcagacacaaggtggtcgtcataccagctgactgcgaagctgctggccttggcctcacattatcagacagacaaattcttactgagaaggtgaacatcatcttccactccgcagccactgttaaatttgacgagcatctgagggctgcattttatacgaacgcacgagccccattacacttgttaagattagcgagggatataaaaaaacttgacgttttgatgcatatatcaacggtgtattcgaattcacatttacaatacgtcgaggagaaatactacccatgtgatatcagctgtgacgagttgcagaaagagatcgacaagatgaccgatgctgaaatagacaaaagtttaccaagacttctcggtgcatggcctaacacatatacttttaccaaggcattagcggagaaagagcttagaacgactgctggggacacaccagttgggattttcagaccagctatagtgacatcaacagcgaacgaacccctcaaatgttggttggacaatatgtacggaccgaatggtgtggcagccggcagtgtgacaggtattttaagaatcctccagtgtgaccaagatgtgacagcagaaatagtgccagttgattacgtcgtcaactgtctgatggtcgcagcagccagtgtgcacgccacttataaaactagtcctccaccagaaccgcctatcttcaactacgtaagctccgtcgagaacaggataacatggggtgattacatgacacaaaatatggcaagagtagacagaaatccattctcaaacgctatatggtacatatcattaacgctgactaaatctgcattcatgtataaattctacatggtatttcttcatttgctaccagctcttttaatggacggactagccacctgcttagggaggaaaccaaagatgctcaaagtttaccgtaagatacacaaattctcttcggtgttatcatatttttgtacgcgagaaataatattctgcaataagcgatcgagggaattgtggcatgacacatcggaagcggatcgaaagatattcccattcagcatggcgaatgttgattggaatgagtattttgatcattacctcgccggaataaggaggtatctgtttaaagagagcgatgcgacattaccacaagccagaatcaaatggaaaagattgtactatctacatcaggtagtaagaataattttcttctgtttcgccatttacgctctctggtcgatgacatcattaatattttaa

SEQ ID NO: 24 - RNA, 1569 nt, Diatraea saccharalis
  source 1..1569: mol_type = other RNA
atggcttggagcaccggtgggactaatgcccacgaatatgtccctgttgcggacttttatgcggacaaatccatcttcgtgaccggtggaacggggtttatgggaaaagtactagtcgaaaaattattaaggagttgtccgaaaatcaagaagatctatctactaatgcgaccgaagcgcggacaagatgtcgcgtcgcgtctcactgagctaacgcagtcaccgctcttcgagacgttgcggaaagacagaccccaagagttgaagaagattgtaccgatcgttggtgatatcactgaaccagaattgggcatcagttccgcggatcaggccatgctctgtcaaaaggtgtcagttgtatttcattcggcggccacagtaaagtttgacgagaaactgaagctctcggtcaccatcaatatgctgggcacgcagcagctggtgcaattgtgtcatcgtatgctcggtttggaggctctagttcatgtttcgaccgcatactgtaactgtgagagggaacgtgttgaggaaacagtatacgcgccgcccgcgcatcctgagcacgtggttacgttagtgcagactttaccagacgagctggtagacagaattacaccagacttagtgggcgaccggccaaatacttacacattcacaaaggctttggcagaagatatgcttataaaggaatgtggcaacttacccgtagcgatcgtgaggccatctatcgtattgtcatctctaagagagcccgttaagggttgggtggacaactggaacggtcccaacggtataatagctgcagtcggcaaaggaatattccgtacgatgcttggcacagggtcgagggtggctgatctagttcccgtcgataccgtcatcaacctgatgatagttagcgcgtggaggacacattcgagacgaggggaaggagtcgtggtatacaactgctgtaccggccaacagaaccccatcacgtggcagcggttcgtcaagaccagcttcaaatatatgcggaaacatccgttcagtgaagtgctctggtaccccggcggtgacataaccagcaatcgtctgaagcatagcgccctcacactcgttcaacatcgtgcgcccgccgtggtgttggatcttgtcgccagagcttcaggaaataagcctttgatgatccgtgttcaaaataaattagaaaaagcggccgcgtgcttggaatactttactacccgccagtggtcgtttgcggacgacaacgttcaagctctctgcgccgctctctcgcaagaagacagaagaacgttcgacttcaacgtgaggaacatcgactgggacgcgtatattgagtcttatgtgttgggaattagacggtttctgttcaaggagagccctgatacgctacccaagtcgagggcgttgatacgaagattacacatagttcacgtcttaacccaagtggcgaccgtgtttttcttatggagatttttattttcccgttccaccgcacttcgcaatatatggcgctacatactcgatttattaacaagagccgcacgtctgcttgccatagcctga

SEQ ID NO: 25 - RNA, 1869 nt, Diatraea saccharalis
  source 1..1869: mol_type = other RNA
atgaaacaatatcgtgcgccgtctatgctgacggcgctttgggtgttgttaatagcgctcccgacagaaactcaacaagtcaacccgatcacatctttcatgaacttcgtaaacgaaggcacaaggcagctcgaccaagaaccaccagaccaagctcgcctccttcccgagtatgacttcatcatagtaggcgctggcacagcaggatgcgttatagccaacagactaacagaaataccagaatggaaagtgttactaatagaagcaggcgtcaacgaaaattatgtcatggacatacccctcgtggccaactatctacaattcaccgaagccaactggaagtacaaaactcaaccgtcggacaggtactgcgctggttttgataacaaacaatgccattggccgcgagggaaagttgtcggtggttccagtgtcttgaactacatgatatacacgagaggctccaaaccagattacgatagctggaaagatatgggcaacgacggctgggcttgggatgacgtcttaccatattttaagaagatagaaaactttaaaattcctaaattcaataattcaaaataccacggaaatgaaggatttctcaacatcgaacacgctccatatcgcagtccagcaagtaaagcgtgggtgaaaggagcgcaacagctcggtttcaagtatggggactataatggcgaagatccagtgagtgtatcatttttacaactctcaatgaaaaacggaacacgacatagctctagccgagcatacttacatcctataaacggtaggaaaaatcttcacatatcaaaagcgagtatggtaacaaaattgatctttgatgacactcaaaccaaagtattgggcgtggaactagaaaaacatggtataaaatataaaatattagcgagcaaagaagtgattctgtctgctggtgcaataaattcccctcaattgttaatgttatccggaattggcccaaaagaccatttagaatcgaagaaaatcaaggtggtcaaagatcttcctgtcggctataacttaatggatcatatagcagcaggagggctacaatttgttattcggtctcaaaacttcagtttgagcacccaatacattttaaatcacttagacatagtatttaaatggatgaagacccacaatggacctctgtctataccaggcggatgcgaagcacttgtttttacagatctaaaagacaaattcaatccaaaaggctggccagacatggaactacttttcataggttctagtttaaacgcagatccgctgttacaatataatttcaatttcgacaaagacatttatagcgacacgtttggacctatgggcaacgcaaacgcttttatggtattcccgatgttgatgcgaccaaagtcaaaaggtagggtaatgttacagagtagaaacccaaaagcaccgccgatattgataccaaactactttgattatgaagaagatttaaaaaagatcgtggaaggaatgaaattggctatagagattacaagacaacctgcaatgaaaaaaataggtgcaaaattgtatgatgttccaattgaagaatgtttgaactatggaccttttgggagtgacgaatacttcgcttgccacgctcaaatgtttactttcacgatataccatcagtgtggaacgtgtaagatgggaattgccgaagacccttcatctgtggtcgactcacgtttaagagttcacggcattgaaaatttaagggtgatagacgctagcataatgcctgaaatagttgcgagtcacacaaatgcaccagtgtatatgatagcggaaaaaggctcagatatgatcaaacaggactggaggaaaatgtaa